(19) United States
(12) Patent Application Publication
(10) Pub. No.: US 2016/0053327 A1
(43) Pub. Date: Feb. 25, 2016

(54) COMPOSITIONS AND METHODS FOR PREDICTION OF CLINICAL OUTCOME FOR ALL STAGES AND ALL CELL TYPES OF NON-SMALL CELL LUNG CANCER IN MULTIPLE COUNTRIES

(71) Applicants: Tiehua Chen, Salt Lake City, UT (US); Luming Chen, Salt Lake City, UT (US); Jing Ling, Salt Lake City, UT (US)

(72) Inventors: Tiehua Chen, Salt Lake City, UT (US); Luming Chen, Salt Lake City, UT (US); Jing Ling, Salt Lake City, UT (US)

(21) Appl. No.: 14/467,002

(22) Filed: Aug. 23, 2014

(51) Int. Cl. C12Q 1/68

(52) U.S. Cl. CPC C12Q 1/6886; C12Q 2600/158; C12Q 2600/118

(57) ABSTRACT

Lung cancer is one of the most commonly diagnosed cancers in the world. While numerous predictive genetic models of non-small cell lung cancer (NSCLC) have been proposed, but many current models fail to accurately predict patient survival when verified by other multiple datasets. Here, we successfully eliminated institutional variations and merged twelve datasets from different institutions to generate a training cohort of 1073 and a testing cohort of 659. From the training cohort, we identified 129 deferentially expressed probes or 95 genes (Table1-2) associated with Lung Cancer.

Here we showed that using seven genes from Table1-2 and combined these genes values with the clinical parameters of age and cancer stage to design the Lung Cancer Prognostic Index (LCPI). Using the LCPI, we were able to differentiate patient populations into low, intermediate, and high risk groups and predict patient survival probabilities for all stages and all cell types of NSCLC at 10 and 15 years. The overall survival probability of low risk group defined by LCPI at 15 years was 65%-100%. Those lung cancer patients were surgical curable. Any post-surgery treatment like ACT (adjuvant chemotherapy) might actually decrease survival probabilities or shorten the life of those patients.

We extensively verified the predictive ability of the LCPI model for overall survival and recurrence free survival using six datasets (n=1665) from five different countries, which included samples of multiple cancer stages and all cell types. Using this model, clinicians would be able to prevent thousands of NSCLC patients from receiving excessive and unnecessary treatments and ultimately prolong their lives.

This research has been published in the first issue of “EbioMedicine” (http://www.ebiomedicine.com/article/S2352-3964%2814%2900014-0/fulltext) which is a high quality peer review journal under editorial leadership of “Cell Press” and “The Lancet”.

### BACKGROUND

Lung cancer is a leading cause of death. In 2008, about 12.7 million cases and 7.6 million deaths were reported worldwide¹. Non-small-cell lung cancer (NSCLC) accounts for 85% of all cases of lung cancer, and includes adenocarcinoma (ADC), squamous cell carcinoma (SCC) and large cell carcinoma (LC). Currently, surgical resection is a common procedure for patients with stage I, II, and certain subsets of stage IIIA NSCLC². For patients with stage II, IIIA, and select stage IB, adjuvant cisplatin-based chemotherapy (ACT) after surgical resection is the standard of care³. However, the effectiveness of using ACT to increase patient survival time remains debatable. In the era of personalized medicine, predictive markers can play a crucial role in helping clinicians to separate patients that may benefit from post-surgical treatments and patients that can be spared the burden of overtreatment.

Gene expression profiles (GEP) are valuable sources of patient data. Since the first publications of GEP for lung cancer in 2001⁴, many studies have proposed predictive models to estimate patient survival time. These models ranged from a single gene to hundreds of genes⁵⁻²¹. Models based on the expression of hundreds of genes is economically impractical in the clinic, and models based on fewer genes have not been verified in different testing cohorts due to small sample size and the variations inherent in data collected from a single institution. Additionally, some authors have truncated data collected over 10 or more years to only 5 years, introducing error in survival predictions and contributing to difficulty in verification. As such, we hypothesize that NSCLC survival time is a quantitative and predictable trait. We have generated a more reliable model by combining multiple datasets obtained from different institutions and different countries to increase the sample size and mitigate the error introduced by institutional biases. We collected 17 publically available NSCLC datasets (Table a), standardized 11 of them by removing batch effects, and then combined them to form a training cohort of 1073 and a testing cohort of 659 patients, which are the largest two GEP datasets of NSCLC in the world. In doing so, we demonstrated how large datasets can be generated, normalized, and analyzed by pooling resources from multiple investigators and provided a formula for converting gene expression datasets from two-channel to single-channel data.

From the training cohort, we identified 129 deferentially expressed probes or 95 genes (Table1-2) associated with Lung Cancer. Additionally, multiple studies indicated that gene expression data combined with clinical parameters can improve the predictive capacity of lung cancer survival models^{9,10}. When we analyzed the training cohort, we not only identified seven gene signatures as independent predictive markers, but also found age and stage to be supplementary independent predictors. We designed the lung cancer prognostic index (LCPI) as a predictive score that accounts for the seven biomarkers as well as age and stage, with lower LCPI scores corresponding to higher survival probabilities. Here, we show that we were able to separate the patient populations in the training and testing cohort into three distinct risk groups using the LCPI model. We used 6 other publically available NSCLC datasets as additional testing cohorts for extensive verification and showed that the LCPI model was able to predict patient survival regardless of lung cancer stage, type or country of origin.

What are needed in the art are methods and assays for identifying a gene expression pattern associated with various risk levels, as well as a method of disease prognosis.

What is also needed in the art is a gene-model developed for assessing outcome for subjects that have, or are at risk for developing, NSCLC. Disclosed herein is such a tool, which utilized multiple independent data sets to confirm that LCPI (lung cancer prognosis index) is able to predict clinical outcome of NSCLC in a given subject.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects and together with the description serve to explain the principles of the invention.

FIG. 1. Comparison of batch effects among multiple datasets of NSCLC before and after COMBAT. a. The expression levels of ACTB showed large batch effects among eight datasets (1: GSE3141, n=111; 2: GSE19188, n=91; 3: GSE37745, n=196; 4: GSE31210, n=226; 5: GSE29013, n=55; 7: GSE19804, n=60; 8: GSE18842, n=46; 9: GSE10245, n=58) of NSCLC in training cohort before COMBAT. b. The batch effects among eight datasets of NSCLC in training cohort have been completely removed by COMBAT. c. There were large batch effects among five healthy lung control or tumor surrounding normal tissues datasets (2: GSE19188, n=65; 4: GSE31210, n=20; 6: GSE1643, n=40; 7: GSE19804, n=60; 8: GSE18842, n=45). d. The batch effects among five healthy lung control or tumor surrounding normal tissues datasets in training cohort have been eliminated by COMBAT. e. There were some batch effects among five datasets (DFCI, HLM, MI, MSKCC and GSE4573, n=659) of NSCLC in testing cohort before COMBAT. f. The batch effects among five datasets of NSCLC in testing cohort were completely eliminated by COMBAT. Bottom, middle, and top lines of each box corresponded to the 25th percentile, the 50th percentile (median), and the 75th percentile, respectively. The caps showed minimum and maximum values excluding outliers.

FIG. 2. The distributions of overall survival time (OST, months) of NSCLC. The histograms showed the frequencies of OST for 306 of deaths in training cohort. The color curves are the fits with three normal distributions. The arrows show the best cutting off values (16m and 60m) for three survival groups.

FIG. 3. Strategies for genes screening. We have performed Siggenes analysis for multiple two-group comparisons (H vs Ca; N vs Ca and poor (OST<16 m) vs good clinical outcome (OST>60 m) and two three-group comparisons (H-vs-N-vs-Ca and poor (OST<16 m) vs good clinical outcome (OST>60 m) vs intermediate subgroup (16 m<OST<60 m). The FDR are less than 0.01 or<0.05. From a total of 54675 probes, we have identified 11571 probes differentially expressed between the two groups (H vs Ca), 10285 probes differentially expressed between N and Ca samples and 1951 probes differentially expressed between poor clinical outcome group and good clinical outcome group. Intersecting the three sets of differentially expressed probes, we have identified 214 common probes (FIG. 3 Right). Among H, N and Ca three groups, we have identified 5779 probes and 4545 differentially expressed probes among different clinical outcome groups. Intersecting the two sets of differentially expressed probes, we have also identified 338 common probes (FIG. 3 Left). Intersecting the two sets of differentially expressed probes from two different strategies, we have identified 129 common probes. There are 95 of common genes (Table 1 and Table 2) differentially expressed excluding repeated probes shared the same gene names among 129 common probes. We have performed univariate analysis (AFT model) for 95 of those genes. For the genes with p value less than 0.01 we have further performed multivariate analysis and Kaplan-Meier analyses. Using 0.05 as p cutting off values, we have finally chosen seven genes (included 5 up-regulated and 2 down-regulated genes, Table b).

FIG. 4. Kaplan-Meier analysis of OS on training cohort. a. Using seven-gene score to predict OS in three stages and three cell types without ACT. b. Using age to predict OS in three stages and three cell types without ACT. The green, blue, black and red lines correspond to the first, second, third and fourth quartiles respectively. c. Using stages to predict OS in three cell types without ACT. The green, blue and red lines correspond to the stage I, II and III separately. d. Using cell types to predict OS in three stages without ACT. The green, blue and red lines correspond to ADC, LC and SCC respectively. e. LCPI defines low, intermediate and high risk subgroups in training cohort without ACT for OS. f. LCPI defines low, intermediate and high risk subgroups in training cohort with ACT for OS. In a, e, and f, green, blue and red lines correspond to low, intermediate and high risk subgroups respectively. The x-axis is the survival time (months), the y-axis is survival probability.

FIG. 5. Effects of ACT or ART on NSCLC in training and testing cohorts and LCPI for RFS. a. The OS probabilities in both ACT (red) and unknown (blue) subgroups were markedly decreased comparing to non-ACT subgroup (green) in training cohort. b. The OS probability in ART (red) subgroup was the lowest comparing to other subgroups in testing cohort. On contrary, the OS probability in non-adjuvant treatment (green) subgroup was the highest. The OS probabilities in ACT (black), ACT+ART (pink) and unknown (yellow) subgroups were lower than non-adjuvant treatment subgroup (green), but higher than ART subgroup (red). c. In low risk subgroup defined by LCPI in training cohort, all the patients in non-ACT subgroup (green) had high up to 100% of survival probabilities at 15 years, but the survival probabilities in ACT (blue) or unknown subgroups were sharply dropped. d. In intermediate risk subgroup defined by LCPI in training cohort, ACT (blue) had no benefit even made it worse at longer follow-up time compared to non-ACT (green). The survival probability in unknown subgroup (red) was severely dropped at any time points. e. In high risk subgroup defined by LCPI in training cohort, the survival probabilities in ACT (blue) and unknown (red) subgroups were similar to non-ACT subgroup (green). The x-axis is the survival time (months), the y-axis is survival probability. f. LCPI defined low, intermediate and high risk subgroups in training cohort for RFS.

FIG. 6. Verification of LCPI in multiple large NSCLC datasets including all stages and all cell types from multiple countries. a. OS, dataset GSE42127, n=176, including two cell types, all stages and 49 ACT, from USA. b. OS, dataset GSE41271, n=274, including seven cell types, all stages and 49 ACT, from USA. c. OS, dataset GSE30219, n=271, including seven cell types, all stages from France. d. OS, Integrated datasets (DFCI, HLM, MI, MSKCC and GSE4573), n=659, including three cell types, three stages (I˜III), 137 ACT and 64 ART, form USA & Canada. e. RFS, dataset GSE8894, n=136, including two cell types and all stages, from South Korea. f. RFS, dataset GSE41271, n=274, including seven cell types, all stages and 49 ACT, from USA. g. OS, two-channel dataset GSE11969, n=149, including five cell types and three stages (I˜III), from Japan. In a, b, c, d, e, f, g, green, blue and red lines correspond to low, intermediate and high risk subgroups defined by LCPI respectively. The x-axis is the survival time (months), the y-axis is survival probability.

### DETAILED DESCRIPTION OF THE INVENTION

Disclosed herein are gene expression panels, sequences and arrays, as well as methods, for assessing prognosis, subgroup type, or survival time of a subject diagnosed with NSCLC, said panel or array consisting of primers or probes or sequences capable of measuring expression levels of a statistically significant number of genes of one or more of the genes identified in Table 1 and Table 2. For example, disclosed are gene expression panels, sequences and arrays, as well as methods, for assessing prognosis, subgroup type, or survival time of a subject diagnosed with NSCLC, said panel or array consisting of primers or probes or sequences capable of measuring expression levels of the genes in Table 1 and Table 2. Also disclosed are diagnostic/prognostic methods, methods of personalized treatment, as well as kits. Also disclosed are methods of discriminating normal, and malignant lung tissue cells in an individual.

All patents, patent applications and publications cited herein, whether supra or infra, are hereby incorporated by reference in their entireties into this application in order to more fully describe the state of the art as known to those skilled therein as of the date of the invention described and claimed herein.

It is to be understood that this invention is not limited to specific synthetic methods, or to specific recombinant biotechnology methods unless otherwise specified, or to particular reagents unless otherwise specified, to specific pharmaceutical carriers, or to particular pharmaceutical formulations or administration regimens, as such may, of course, vary. It is also to be understood that the terminology used herein is for the purpose of describing particular embodiments only and is not intended to be limiting.

### Definitions and Nomenclature

The terminology used herein is for the purpose of describing particular embodiments only and is not intended to be limiting.

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

By ‘sample” is meant an patient; a tissue or organ from an patient; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

By “overall survival” is the length of time from the date of surgery treatment for the lung cancer, that patients after surgery are still alive. In a clinical trial, measuring the overall survival is one way to see how well a new treatment works. It also called OS.

By “relapse-free survival” or “recurrence-free survival” or “disease-free survival” is the length of time after primary treatment (surgery) for a lung cancer ends that the patient survives without any signs or symptoms of lung cancer. Also it called RFS or DFS, which is totally different from OS.

By “modulate” is meant to alter, by increasing or decreasing.

By “normal subject” is meant an individual who does not have NSCLC.

The phrase “nucleic acid” or ‘sequences” as used herein refers to a naturally occurring or synthetic oligonucleotide or polynucleotide or any sequence, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

By “treat” is meant to administer a compound or molecule or a surgery to a subject, such as a human or other mammal (for example, an animal model), that has a condition or disease, such as NSCLC, an increased susceptibility for developing such a disease, in order to prevent or delay a worsening of the effects of the disease or condition, or to partially or fully reverse the effects of the disease. To “treat” can also refer to non-pharmacological methods of preventing or delaying a worsening of the effects of the disease or condition, or to partially or fully reversing the effects of the disease. For example, “treat” is meant to mean a course of action to prevent or delay a worsening of the effects of the disease or condition, or to partially or fully reverse the effects of the disease other than by administering a compound.

By “prevent” is meant to minimize the chance that a subject who has susceptibility for developing disease such as NSCLC will develop such a disease, or one or more symptoms associated with the disease.

By “probe,” “primer,” “oligonucleotide” or “sequences” is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for c-Met nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By ‘specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65oC, or a buffer containing 48% formamide, 4.8×SSC, 0.2 M Tris-Cl, pH 7.6, 1× Denhardt's solution, 10% dextran sulfate, and 0.1% SDS, at a temperature of 42oC. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

The nucleic acids, such as, the polynucleotides described herein, can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 3rd Edition (Cold Spring Harbor Laboratory Press, Cold Spring Harbor, N.Y., 2001) Chapters 5, 6) to purely synthetic methods, for example, by the cyanoethyl phosphoramidite method using a Milligen or Beckman System 1 Plus DNA synthesizer. Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### Compositions

NSCLC are ultimately fatal outcome for most patients. The five-year survival rate for lung cancer continues to be poor at only about 8-15%. However the OS time are varying from 0 to 180 months. Thus, there are distinct clinical subgroups of NSCLC, and modern molecular tests may provide help in identifying these entities.

Disclosed herein are gene expression panels, sequences and arrays indicative of survival time of a subject diagnosed with NSCLC, said panel or array consisting of primers or probes or sequences capable of measuring expression levels of a statistically significant number of genes of Table 1 and Table 2. For example, in one embodiment, the gene expression panel or array consists of primers or probes or sequences capable of measuring expression levels of the genes in Table 1 and Table 2. This expression panel plus age and stages is herein referred to as the NSCLC Survival prediction Index (LCPI). LCPI was developed from GEP data sets of 60 of healthy lung tissue cells (H), 170 of normal surrounding tissue cells (N), and 843 of NSCLC. COMBAT package in R/bioconductor was used to remove batch effects and siggenes package was used to screen significantly expressed genes which results were then analyzed by Kaplan-Meier analysis. The disease prognostic power of LCPI was evaluated with multiple independent data sets of other 1665 patients both for OS or RFS.

Many genes associated with low-risk disease in NSCLC are identified, and these are found in Table 1 and Table 2. These are sometimes referred to herein as “the biomarkers” or “the nucleic acids or polypeptides disclosed herein.” Survival analysis showed that a low LCPI signature was associated with longer survival. Applying LCPI to independent data sets, 5-30% of patients were classified as low-risk, with a survival probability of 65%-100% at 15 years. Multiple clinical parameters confirmed significant correlation between low and high-risk subgroups defined by LCPI. When previously published models were applied to the same data sets it was observed that LCPI model retained the best prognostic value.

Disclosed herein is a gene expression panel, sequence or array indicative of survival time of a subject diagnosed with NSCLC, said panel, sequence or array consisting of primers or probes or sequences capable of measuring expression levels of a statistically significant number of genes of one or more of the genes identified in Table 1 and Table 2. The sequences of one or more of the genes can be found in the GenBank database.

The profile can be provided in the form of a graph or tree view. The profile of the expression levels of the genes can be used to compute a statistically significant value based on differential expression of the group of genes, wherein the computed value correlates to a diagnosis for a subgroup of NSCLC. The variance in the obtained profile of expression levels of the said selected genes or gene expression products (including RNA or Protein) can be either up regulated or down regulated as compared to a control.

The gene expression panel, sequence or array can consist of primers or probes or sequences capable of detecting one or more genes disclosed in Table 1 and Table 2. Examples of primers or probes or sequences capable of detecting one or more genes include, but are not limited to the primer and probes.

Also disclosed are diagnostic kits containing probes or primers or sequences for measuring the expression of one or more of the genes disclosed herein. For example, disclosed are diagnostic kits containing probes or primers or sequences for measuring the expression of one or more of the genes in Table 1 and Table 2.

Disclosed herein do solid supports comprise one or more primers, probes, polypeptides, sequences or antibodies capable of hybridizing or binding to one or more of the genes found in Table 1 and Table 2. Solid supports are solid-state substrates or supports with which molecules, such as analytes and analyte binding molecules can be associated. Analytes, such as calcifying nano-particles and proteins, can be associated with solid supports directly or indirectly. For example, analytes can be directly immobilized on solid supports. Analyte capture agents, such a capture compounds, can also be immobilized on solid supports.

The term “differentially expressed” or “differential expression,” as well as the term “variant,” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example as measured by the amount of messenger RNA transcript and/or the amount of protein in a sample as compared with the measurable expression level of a given biomarker in a control. In one embodiment, the differential expression can be compared using the score or ratio of the level of expression of a given biomarker or biomarkers (such as the genes found in Table 1 and Table 2) as compared with the expression level of the given biomarker or biomarkers of a control, wherein the score or ratio is not equal to that of control. For example, an RNA or protein is differentially expressed if the score or ratio of the level of expression in a first sample as compared with a second sample is greater than or less than control. For example, a score or ratio of greater than 1, 1.2, 1.5, 1.7, 2, 3, 3, 5, 10, 15, 20 or more, or a score or ratio less than 1, 0.8, 0.6, 0.4, 0.2, 0.1, 0.05, 0.001 or less. In another embodiment the differential expression is measured using p-value. For instance, when using p-value, a biomarker is identified as being differentially expressed as between a first sample and a second sample when the p-value is less than 0.05, preferably less than 0.01, more preferably less than 0.005, even more preferably less than 0.001, the most preferably less than 0.0001.

The term “similarity in expression” as used herein means that there is no or little difference in the level of expression of the biomarkers between the test sample and the control or reference profile. For example, similarity can refer to a fold difference compared to a control. In one example, there is no statistically significant difference in the level of expression of the biomarkers.

The term “most similar” in the context of a reference profile refers to a reference profile that is associated with a clinical outcome that shows the greatest number of identities and/or degree of changes with the subject profile.

The phrase “determining the expression of biomarkers” as used herein refers to determining or quantifying RNA or proteins or protein activities or protein-related metabolites expressed by the genes disclosed herein. The term “RNA” includes mRNA transcripts, and/or specific spliced or other alternative variants of mRNA, including anti-sense products. The term “RNA product of the biomarker” as used herein refers to RNA transcripts transcribed from the biomarkers and/or specific spliced or alternative variants. In the case of “protein”, it refers to proteins translated from the RNA transcripts transcribed from the biomarkers. The term “protein product of the biomarker” refers to proteins translated from RNA products of the biomarkers.

A person skilled in the art will appreciate that a number of methods can be used to detect or quantify the level of RNA products of the biomarkers within a sample; including arrays, such as microarrays, RT-PCR (including quantitative RT-PCR), nuclease protection assays and Northern blot analyses.

Accordingly, in one example, the biomarker expression levels are determined using arrays, optionally microarrays, RT-PCR, optionally quantitative RT-PCR, nuclease protection assays, Northern blot analyses, RNA sequence or genome sequence.

A form of solid support is an array. Another form of solid support is an array detector. An array detector is a solid support to which multiple different capture compounds or detection compounds have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A form for a solid-state substrate is a microtiter dish, such as a standard 96-well type. In preferred embodiments, a multiwell glass slide can be employed that normally contain one array per well. This feature allows for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array generally can have one type of component (that is, all the components at that location are the same). Each location will have multiple copies of the component. The spatial separation of different components in the array allows separate detection and identification of the polynucleotides or polypeptides disclosed herein.

It is not required that a given array be a single unit or structure. The set of compounds may be distributed over any number of solid supports. For example, at one extreme, each compound may be immobilized in a separate reaction tube or container, or on separate beads or micro particles. Different modes of the disclosed method can be performed with different components (for example, different compounds specific for different proteins) immobilized on a solid support.

Some solid supports can have capture compounds, such as antibodies, attached to a solid-state substrate. Such capture compounds can be specific for calcifying nano-particles or a protein on calcifying nano-particles. Captured calcifying nano-particles or proteins can then be detected by binding of a second, detection compound, such as an antibody. The detection compound can be specific for the same or a different protein on the calcifying nano-particle.

Methods for immobilizing nucleic acids, peptides or antibodies (and other proteins) to solid-state substrates are well established. Immobilization can be accomplished by attachment, for example, to aminated surfaces, carboxylated surfaces or hydroxylated surfaces using standard immobilization chemistries. Antibodies can be attached to a substrate by chemically cross-linking a free amino group on the antibody to reactive side groups present within the solid-state substrate. For example, antibodies may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS3) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120 oC for 4 hrs. Thiol-derivatized slides are activated by immersing in a 0.5 mg/ml solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each of the components (compounds, for example) immobilized on the solid support preferably is located in a different predefined region of the solid support. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components preferably are immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

Optionally, at least one address on the solid support can be a probe specific for one or more of the genes disclosed in Table 1 or Table 2. Disclosed are solid supports where at least one address is the sequences or portion of sequences set forth in any of the peptide sequences disclosed herein. Solid supports can also contain at least one address is a variant of the sequences or part of the sequences set forth in any of the nucleic acid sequences disclosed herein. Solid supports can also contain at least one address is a variant of the sequences or portion of sequences set forth in any of the peptide sequences disclosed herein.

In addition, the genes described herein may be used as markers for presence or progression of NSCLC. The methods and assays described elsewhere herein may be performed over time, and the change in the level of reactive polypeptide(s) or polynucleotide(s) evaluated. Assays can be performed prior to, during, or after a treatment protocol.

As noted herein, to improve sensitivity, multiple genes may be assayed within a given sample. Binding agents specific for different proteins, antibodies, nucleic acids thereto provided herein may be combined within a single assay. Further, multiple primers or probes may be used concurrently. The selection of receptors may be based on routine experiments to determine combinations that results in optimal sensitivity. To assist with such assays, specific biomarkers can assist in the specificity of such tests. As such, disclosed herein is a biomarker, wherein the biomarker is capable of binding to or hybridizing with a metabolite detecting, a gene or peptide as disclosed herein.

According to a further aspect, there is provided a computer implemented product for predicting a prognosis or classifying a subject with NSCLC comprising (a) a means for receiving values corresponding to a subject expression profile in a subject sample; and (b) a database comprising a reference expression profile associated with a prognosis, wherein the subject biomarker expression profile and the biomarker reference profile each have at least three values representing the expression level of at least one biomarker selected from Table 1 and Table 2 implemented product selects the biomarker reference expression profile most similar to the subject biomarker expression profile, to thereby predict a prognosis or classify the subject.

Preferably, a computer implemented product described herein is for use with a method described herein.

According to a further aspect, there is provided a computer implemented product for determining therapy for a subject with NSCLC comprising: (a) a means for receiving values corresponding to a subject expression profile in a subject sample; and (b) a database comprising a reference expression profile associated with a therapy, wherein the subject biomarker expression profile and the biomarker reference profile each have at least one value, the at least one value representing the expression level of at least one biomarker selected from Table 1 and Table 2 wherein the computer implemented product selects the biomarker reference expression profile most similar to the subject biomarker expression profile, to thereby predict the therapy.

According to a further aspect, there is provided a computer readable medium having stored thereon a data structure for storing a computer implemented product described herein.

Preferably, the data structure is capable of configuring a computer to respond to queries based on records belonging to the data structure, each of the records comprising: (a) a value that identifies a biomarker reference expression profile of at least one gene selected from Table 1 and Table 2, (b) a value that identifies the probability of a prognosis associated with the biomarker reference expression profile.

According to a further aspect, there is provided a computer system comprising (a) a database including records comprising a biomarker reference expression profile of at least one gene selected from Table 1 and Table 2 associated with a prognosis or therapy; (b) a user interface capable of receiving a selection of gene expression levels of the at least one gene for use in comparing to the biomarker reference expression profile in the database; (c) an output that displays a prediction of prognosis or therapy according to the biomarker reference expression profile most similar to the expression levels of the at least one gene.

In a further aspect, the application provides computer programs and computer implemented products for carrying out the methods described herein. Accordingly, in one embodiment, the application provides a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the methods described herein.

### Methods

The disclosed gene and peptides (as found in Table 1 and Table 2) can be used in a variety of different methods, for example in prognostic, predictive, diagnostic, and therapeutic methods and as a variety of different compositions.

Also disclosed is a method of diagnosing or assessing a subject's susceptibility to develop NSCLC (also referred to as a prognosis for a subject) comprising: extracting RNA from a biological sample of said subject containing cancer cells; generating cDNA from said RNA; amplifying said cDNA with probes or primers for genes, gene sequences or gene expression products, wherein said genes or gene expression products are selected from a statistically significant number of genes or gene expression products of one or more genes identified in one or more of the Tables disclosed herein (such as Table 1 and Table 2); and obtaining from said amplified cDNA a profile of the expression levels of the selected genes or gene expression products in said sample; and diagnosing or assessing a subject's prognosis upon a variance in the obtained profile of expression levels of the said selected genes or gene expression products in said subject's sample from the same selected genes or gene expression products of a control gene expression profile from a similar biological sample of a healthy subject, or diagnosing or assessing a subject's prognosis upon a similarity in the obtained profile of expression levels of said selected genes or gene expression products in said subject's sample to the same selected genes or gene expression products in a gene expression profile characteristic of a subject with NSCLC.

Further disclosed is a method for prognosis of NSCLC in a mammalian subject comprising extracting RNA from a biological sample containing lung cancer cells of the subject; generating cDNA from said RNA; amplifying said cDNA with probes or primers for a statistically significant number of genes or gene expression products of Table 1 and Table 2; obtaining from said amplified cDNA the expression levels of said genes or gene expression products in said sample; prognosis of NSCLC based upon a variance in the pattern of obtained expression levels of the said genes or gene expression products that form a gene expression profile characteristic of NSCLC in said subject's sample.

Also disclosed is a method of assessing a subject's susceptibility to develop NSCLC, the method comprising: amplifying cDNA from a biological sample containing lung cancer cells of the subject to obtain expression levels of a statistically significant number of genes or gene expression products obtained from said sample, wherein said genes or gene expression products are selected from a statistically significant number of genes or gene products of Table 1 and Table 2, thereby assessing a subject's susceptibility to develop NSCLC based on a change in a profile of expression levels between said selected genes or gene products of said sample from the same selected genes or gene products of a control healthy expression profile, wherein said change indicates a subject's susceptibility to develop NSCLC.

As described herein, disclosed are methods of detecting NSCLC in a sample comprising determining the expression level of one or more genes in a sample and comparing those expression levels to the expression levels of a normal sample, wherein the expression level of one or more metabolite detecting genes or peptides is increased or decreased by 10%, 15%, 20%, 25%, 30%, 35%, 40%, 45%, 50%, 55%, 60%, 65%, 70%, 75%, 80%, 85%, 90%, 95%, or 100% when compared to the expression level of a “normal” subject is indicative of a NSCLC. In addition, the expression level of one or more genes or peptides as found in Table 1 can be increased or decreased by 10%, 15%, 20%, 25%, 30%, 35%, 40%, 45%, 50%, 55%, 60%, 65%, 70%, 75%, 80%, 85%, 90%, 95%, or 100% when compared to the expression level of a “normal” subject is indicative of a pathological condition.

An increase or decrease in the expression level of the genes or peptides disclosed herein is not always required to indicate NSCLC. There can be signature patterns of increased or decreased expression levels of one or more of the genes or peptides.

For example, an increase in the expression level of some genes in Table 1 and Table 2 can indicate NSCLC.

Further disclosed is a method of discriminating low and high risk in an individual, comprising the steps of: obtaining mRNA expression patterns of a statistically significant number of genes or gene products of Table 1 and Table 2 in a sample of lung tissue cells from the individual; performing a discriminant analysis on the gene expression patterns to compute a discriminant score; and comparing the discriminant score to a predictive cutoff value statistically determined from a control model of the genes; wherein a score below the cutoff value is indicative that the NSCLC patients are at low risk and a score above the cutoff is indicative that the patients are at high risk.

A progressive deregulation of multiple components of the signaling complex can be associated with disease progression from normal lung tissue cells to NSCLC.

Disclosed is a method of diagnosing or assessing a subgroup of NSCLC in a subject, the method comprising: extracting RNA from a biological sample of said subject containing cancer cells; generating cDNA from said RNA; amplifying said cDNA with probes or primers for genes or gene expression products, wherein said genes or gene expression products are selected from one or more genes identified in one or more of the Tables disclosed herein; obtaining from said amplified cDNA a profile of the expression levels of the selected genes or gene expression products in said sample; and diagnosing or assessing a subject's subgroup based upon a variance in the obtained profile of expression levels of the said selected genes or gene expression products in said subject's sample from the same selected genes or gene expression products of a control gene expression profile from a similar biological sample of a healthy subject, or diagnosing or assessing a subject's subgroup based upon a similarity in the obtained profile of expression levels of said selected genes or gene expression products in said subject's sample to the same selected genes or gene expression products in a gene expression profile characteristic of a subject with NSCLC.

Subgroups of NSCLC include low, intermediate and high risk. The panels and methods described herein have defined 5-30% of low risk patients in NSCLC, 50-60% of intermediate risk subgroups. The panels and methods described herein showed that the panels and methods described herein are able to separate low-risk (P<0.01) and high-risk subgroups (P<0.01) from the intermediate-risk population.

“Survival time” or “survival rate” or “survival probability” indicates the likelihood for survival of the disease for a specific period of time after the diagnosis of a subject or after surgery. For example, this can refer to a five year NSCLC survival rate, meaning the chance that a given individual will survive 5 years from the time of their initial diagnosis or surgery, or from another given point. Along with the genes analysis described herein, other factors that can affect the survival rate, which can also be considered when calculating the rate, include the stage of NSCLC when diagnosed, and the subject's age.

“Prognosis” refers to a clinical outcome group such as a poor survival group (high risk) or a good survival group (low risk) associated with a NSCLC subtype which is reflected by a reference profile, or reflected by an expression level of the LCPI signature disclosed herein. The prognosis provides an indication of disease progression and includes an indication of likelihood of death due to NSCLC. In one embodiment the clinical outcome class includes a good survival group an intermediate group and a poor survival group.

The term “prognosis” or “classifying” as used herein means predicting or identifying the clinical outcome group that a subject belongs to according to the subject's similarity to a reference profile or LCPI signature associated with the prognosis. For example, prognosis or classifying comprises a method or process of determining whether an individual with NSCLC has a good or poor survival outcome, or grouping an individual with NSCLC into a good survival group or a poor survival group. Also included is determining the risk level of developing NSCLC, in a subject that has not been diagnosed with the disease.

The term “good survival” as used herein refers to an increased chance of survival as compared to patients in the “poor survival” group. For example, the genes in Table 1 and Table 2 can be used to prognosis or classify subjects into a “good survival group”. These patients are at a lower risk of death. Good survival, as used herein, is defined as being expected to have a great chance (>55%) to survive for fifteen years or more.

The term “poor survival” as used herein refers to an increased risk of death as compared to subjects in the “good survival” group. For example, the genes in Table 1 and Table 2 can be used to prognosis or classify subjects into a “poor survival group”. These patients are at greater risk of death. Poor survival, as used herein, is defined as being expected to have a low chance (<45%) to survive for five year.

In one example, the variance in the obtained profile of expression levels of the said selected genes or gene expression products in said subject's sample can be used to determine whether a subject is at a low, intermediate, or high risk of death. The terms “low, intermediate, and high” are relative terms, which can mean, for example, that the subject is at low risk (35% or less chance of death), intermediate (35%-65% chance of death) or high risk (65% chance or greater of death).

The sample derived from the subject to carry out the array test disclosed herein can be derived from a variety of sources, but is typically derived from lung tissue cells tumor cells.

The variance in the obtained profile of expression levels of the said selected genes or gene expression products in said subject's sample can be used to determine the type of treatment, or combination of treatments, that the subject should receive. Examples of treatments typically given to subjects in high risk groups diagnosed with NSCLC include, but are not limited to:

Abitrexate (Methotrexate)

Abraxane (Paclitaxel Albumin-stabilized Nanoparticle Formulation)

Afatinib Dimaleate

Alimta (Pemetrexed Disodium)

Avastin (Bevacizumab)

Bevacizumab

Carboplatin

Cisplatin

Crizotinib

Docetaxel

Doxorubicin

Erlotinib Hydrochloride

Etoposide

Folex (Methotrexate)

Folex PFS (Methotrexate)

Gefitinib

Gilotrif (Afatinib Dimaleate)

Gemcitabine Hydrochloride

Gemzar (Gemcitabine Hydrochloride)

Iressa (Gefitinib)

Methotrexate

Methotrexate LPF (Methotrexate)

Mexate (Methotrexate)

Mexate-AQ (Methotrexate)

Paclitaxel

Paclitaxel Albumin-stabilized Nanoparticle Formulation

Paraplat (Carboplatin)

Paraplatin (Carboplatin)

Pemetrexed Disodium

Platinol (Cisplatin)

Platinol-AQ (Cisplatin)

Tarceva (Erlotinib Hydrochloride)

Taxol (Paclitaxel)

Taxotere (Docetaxel)

Vinorelbine

Xalkori (Crizotinib).

Radiation therapy is yet another option. These treatments can be used alone or in combination, and as stated above, the results of the LCPI signature can help determine the subgroup for treatment.

Also disclosed is a method for treating NSCLC in an individual, comprising the step of: modulating expression of one or more genes identified in one or more of the Tables disclosed herein; thereby altering differential expression of the NSCLC genes to treat the individual. Also disclosed herein are methods that can be used to evaluate the efficacy of various clinical interventions.

The term “modulate”, as used herein, refers to a change or an alteration in the biological activity of a gene or a gene product, such as a polypeptide. Modulation may be an increase or a decrease in expression level or peptide activity, a change in binding characteristics, or any other change in the biological, functional or immunological properties of the nucleic acid or polypeptide. In one example, some genes can be upregulated, and others downregulated, simultaneously. For example, in some aspects an increase in the expression level or upregulation of some genes in Table 1 and Table 2 correlates to a diagnosis or prognosis for a subgroup of NSCLC. In some aspects a decreased expression or down regulation of some genes in Table 1 and Table 2 correlates to a diagnosis or prognosis for a subgroup of NSCLC. In some aspects, a combination of an increase in the expression level or upregulation of some genes in Table 1 and Table 2 and a decreased expression or down regulation of some genes in Table 1 and Table 2 correlates to a diagnosis or prognosis for a subgroup of NSCLC.

Disclosed herein are functional nucleic acids that can interact with the disclosed receptor. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, ribozymes, triplex forming molecules, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of polynucleotide sequences disclosed herein or the genomic DNA of the polynucleotide sequences disclosed herein or they can interact with the polypeptide encoded by the polynucleotide sequences disclosed herein. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, aptamers, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10-6, 10-8, 10-10, or 10-12. A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437 each of which is herein incorporated by reference in its entirety for their teaching of modifications and methods related to the same.

Disclosed are aptamers that interact that interact with the disclosed nucleic acids and could thus inhibit the expression of such Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10-12 M. It is preferred that the aptamers bind the target molecule with a kd less than 10-6, 10-8, 10-10, or 10-12. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a kd with the target molecule at least 10, 100, 1000, 10,000, or 100,000 fold lower than the kd with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Disclosed are ribozymes that interact with the disclosed nucleic acids and could thus inhibit the expression of such. Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Disclosed are triplex forming functional nucleic acid molecules that interact with the disclosed nucleic acids and could thus inhibit the expression of such. Triplex forming functional nucleic acid molecules are molecules that can interact with either double-stranded or single-stranded nucleic acid. When triplex molecules interact with a target region, a structure called a triplex is formed, in which three strands of DNA are forming a complex dependant on both Watson-Crick and Hoogsteen base-pairing. Triplex molecules are preferred because they can bind target regions with high affinity and specificity. It is preferred that the triplex forming molecules bind the target molecule with a kd less than 10-6, 10-8, 10-10, or 10-12. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

Disclosed are external guide sequences that form a complex with the disclosed nucleic acids and could thus inhibit the expression of such. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

Disclosed are polynucleotides that contain peptide nucleic acids (PNAs) compositions that interact with the disclosed nucleic acids and could thus inhibit the expression of such. PNA is a DNA mimic in which the nucleobases are attached to a pseudopeptide backbone (Good and Nielsen, Antisense Nucleic Acid Drug Dev. 1997; 7(4) 431-37). PNA is able to be utilized in a number of methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 June; 15(6):224-9). As such, in certain embodiments, one may prepare PNA sequences that are complementary to one or more portions of an mRNA sequence based on the disclosed polynucleotides, and such PNA compositions may be used to regulate, alter, decrease, or reduce the translation of the disclosed polynucleotides transcribed mRNA, and thereby alter the level of the disclosed polynucleotide's activity in a host cell to which such PNA compositions have been administered.

PNAs have 2-aminoethyl-glycine linkages replacing the normal phosphodiester backbone of DNA (Nielsen et al., Science Dec. 6, 1991; 254(5037):1497-500; Hanvey et al., Science. Nov. 27, 1992; 258(5087):1481-5; Hyrup and Nielsen, Bioorg Med Chem. 1996 January; 4(1):5-23). This chemistry has three important consequences: firstly, in contrast to DNA or phosphorothioate oligonucleotides, PNAs are neutral molecules; secondly, PNAs are achirial, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. 1995 April; 3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. 1995 April; 3(4):437-45; Petersen et al., J Pept Sci. 1995 May-June; 1(3):175-83; Orum et al., Biotechniques. 1995 September; 19(3):472-80; Footer et al., Biochemistry. Aug. 20, 1996; 35(33): 10673-9; Griffith et al., Nucleic Acids Res. Aug. 11, 1995; 23(15):3003-8; Pardridge et al., Proc Natl Acad Sci USA. Jun. 6, 1995; 92(12):5592-6; Boffa et al., Proc Natl Acad Sci USA. Mar. 14, 1995; 92(6):1901-5; Gambacorti-Passerini et al., Blood. Aug. 15, 1996; 88(4):1411-7; Armitage et al., Proc Natl Acad Sci USA. Nov. 11, 1997; 94(23):12320-5; Seeger et al., Biotechniques. 1997 September; 23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993; 65(24):3545-9) and Jensen et al. (Biochemistry. Apr. 22, 1997; 36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore” technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

In addition, antibodies to the proteins disclosed herein can be used to inhibit the function of the receptors, for example, isolated antibodies, antibody fragments and antigen-binding fragments thereof. Optionally, the isolated antibodies, antibody fragments, or antigen-binding fragment thereof can be neutralizing antibodies. The antibodies, antibody fragments and antigen-binding fragments thereof disclosed herein can be identified using the methods disclosed herein.

The term “antibodies” is used herein in a broad sense and includes both polyclonal and monoclonal antibodies. In addition to intact immunoglobulin molecules, disclosed are antibody fragments or polymers of those immunoglobulin molecules, and human or humanized versions of immunoglobulin molecules or fragments thereof, as long as they are chosen for their ability to interact with the polypeptides disclosed herein. As used herein, the term “antibody” or “antibodies” can also refer to a human antibody or a humanized antibody.

“Antibody fragments” are portions of a complete antibody. A complete antibody refers to an antibody having two complete light chains and two complete heavy chains. An antibody fragment lacks all or a portion of one or more of the chains. Examples of antibody fragments include, but are not limited to, half antibodies and fragments of half antibodies. A half antibody is composed of a single light chain and a single heavy chain. Half antibodies and half antibody fragments can be produced by reducing an antibody or antibody fragment having two light chains and two heavy chains. Such antibody fragments are referred to as reduced antibodies. Reduced antibodies have exposed and reactive sulfhydryl groups. These sulfhydryl groups can be used as reactive chemical groups or coupling of biomolecules to the antibody fragment. A preferred half antibody fragment is a F(ab). The hinge region of an antibody or antibody fragment is the region where the light chain ends and the heavy chain goes on.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing from the scope and spirit of this invention. All changes, modifications, and variations coming within the meaning and range of equivalency of the claims are to be considered within their scope.

### EXAMPLES

### Example 1

### Identification of Low-Risk Patients in NSCLC (Prediction of Clinical Outcome for All Stages and Multiple Cell Types of Non-small Cell Lung Cancer in Five Countries Using Lung Cancer Prognostic Index, EBiomedicine, 1(1), 2014, DOI: http://dx.doi.org/10.1016/j.ebiom.2014.10.012)

Design and Methods

GEP Data Collection and Grouping

We collected 17 publically available GEP datasets (n=2738) with clinical parameters from the Gene Expression Omnibus and the National Cancer Institute (GSE26939²² added breast cancer cells as reference was excluded from our studies). As we needed both the GEP data as well as the corresponding clinical parameters, any dataset that did not release or contain either type of data was excluded from our study. The gene expression data was obtained from tumor tissue after surgical resection, and thus we limited our analysis to patients for whom surgical resection is a viable option. Although the analysis is not shown in this paper, we did explore the effect of prior grouping variables. Most of the data in the 17 studies have similar age range, similar gender distribution, and similar death ratios. As a result of the parameters of the original studies, none of the patients receive preoperative chemotherapy. There were a total of 230 control samples. According to the power calculations, to attain 90% power with a significance level of 0.05 and effect size of 0.25, we needed a NSCLC patient sample size of 630. We set nine datasets performed by platform GPL570 (including 54675 probes) as training cohort (n=843). Since GSE30219¹⁹ was the largest single study including all cancer stages and all cancer cell types, we used it as a testing cohort in combination with GSE8894¹⁰, which only contained recurrence-free survival (RFS) data. Six other datasets collected on different platforms were also used for verification 6, 8, 9, 13, 20, 21. We downloaded all available original CEL files and normalized them with Robust Multichip Average from Affymetrix Expression Console.

Combining Nine Datasets in Training Cohort and Three Datasets in Testing Cohort

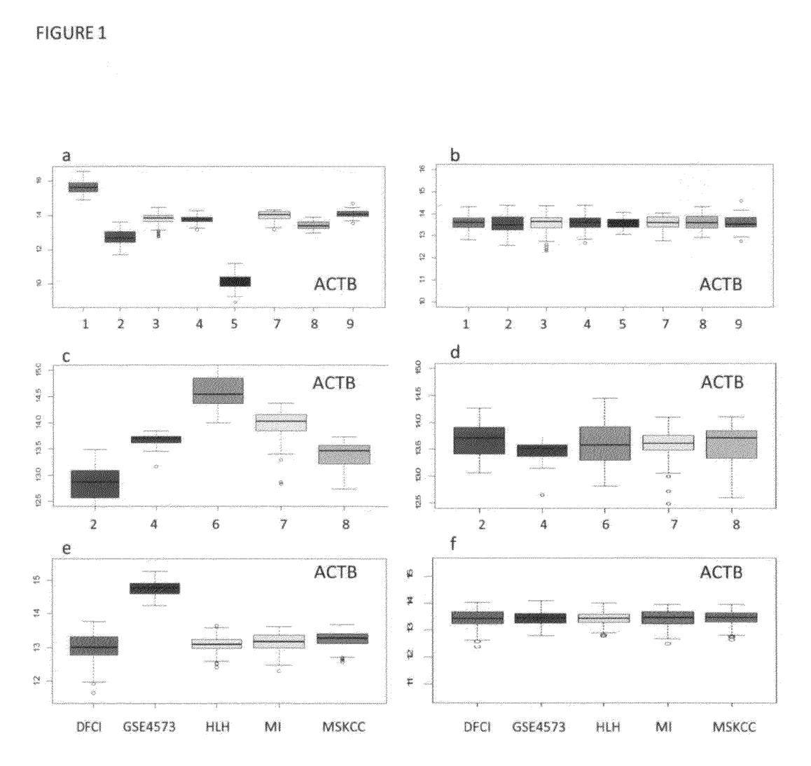

The optimal way of grouping the patient data was to combine all 2738 available samples together and randomize them into two groups: the training cohort and the testing cohort. However, due to the fact that the available datasets were performed on different platforms and contained batch effects, we were compelled to adopt another approach. Although the platform was the same for some datasets, it was impossible to combine them directly due to large batch effects among different datasets (FIG. 1a, c, e). To remove these batch effects, we decided to use COMBAT because it outperformed other available methods²³. Using the COMBAT methodology described previously in Chen, C. et al., we standardized the nine datasets we combined for the training cohort²³. Similarly we combined three GPL96 (22283 probes) datasets for the largest testing cohort. GSE42127²¹ and GSE41271²⁰ were obtained with platform GPL6884 (48803 probes), and to avoid loss of any gene information, we did not perform data merging among different platforms.

Significance Analysis of Differentially Expressed Genes

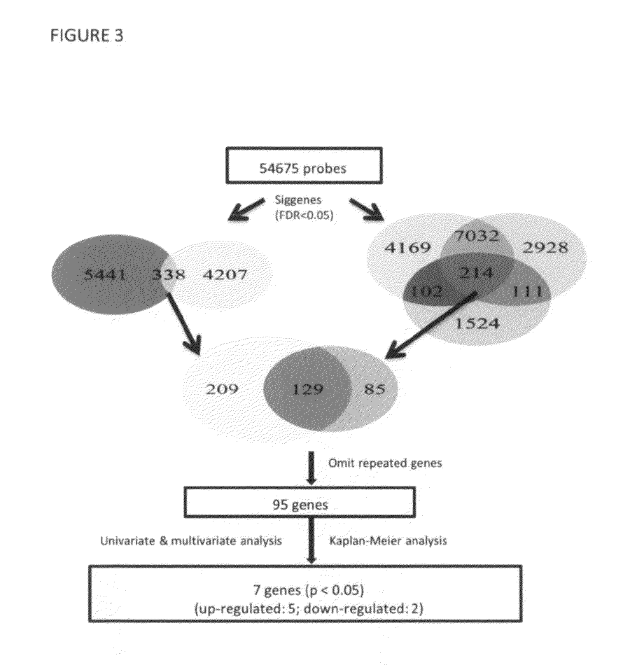

Siggenes was used to identify the differentially expressed genes as previously described²⁴. Since multiple two-group comparisons may introduce some errors, we further compared the three groups simultaneously, and then found the genes expression differences that were common to all comparisons (FIG. 3).

Univariate & Multivariate Analyses (Accelerated Failure Time Model, AFT)

While some studies published overall survival (OS) data that exceeded 5 years of follow-up^{18, 25}, others truncated the data at 5 years^{8, 9, 12, 17, 19}. To generate a more reliable model, we analyzed all available data. The drawback of OS data is that as time passes it can be influenced by many other factors than the cancer itself. To account for the effect of time on OS, we used the AFT model for univariate & multivariate analyses.

Kaplan-Meier Analysis

Kaplan-Meier curve takes into account right-censoring, and all of the NSCLC datasets were right-censored data. We performed Kaplan-Meier analyses and chi-square (X²) tests were used to determine significant differences in R.

Converting Data from Two Channels to Single Channel

There was only one dataset (GSE11969⁶) in testing cohort which was performed with Agilent's two-channel array GPL7015. Two-channel array introduced a reference RNA (labeled with Cyanine-3: Cy3) to compare the samples (labeled with Cyanine-5: Cy5) and exported the ratios of Cy5/Cy3 as follows:

$$\begin{matrix}Etwo = log 10 ( Cy 5 / Cy 3 ) \\ = log 10 ( ( GeneXNSCLC ) / ( GeneXreference ) ) \\ = log 10 ( GeneXNSCLC ) - log 10 ( GeneXreference )\end{matrix} \qquad ( 1 )$$

All single channel data are transformed into log2 values:

E_{single}=log₂(GeneX_{NSCLC})=log₁₀(GeneX_{NSCLC})/log₁₀2 (2)

Combine function (1) and (2):

E_{single}=(E_{two}+log₁₀(GeneX_{reference}))/log₁₀2 (3)

Where E_{two} was normalized log₁₀ ratio of Cy5/Cy3 representing sample/reference. E_{single} was normalized log₂ values of intensity only representing sample. GeneX_{NSCLC} was intensity value of sample. GeneX_{reference} was intensity value of reference RNA.

In GSE11969, total RNA from 20 lung cell lines representing all major histological types of NSCLC was reference. We were able to use the mean expression value of any gene from one-channel of NSCLC cell lines to estimate the log₁₀ (GeneX_{reference}). Using function (3), it was easy to transform all log₁₀ ratios of two-channel data into one-channel data.

Results

Removal of Large Batch Effects

The housekeeping gene Beta-actin (ACTB) expression showed that there were large batch effects due to institutional variations among the training datasets (FIG. 1a, c). The biggest variation was observed between the datasets of study 1 (GSE3141⁵) and study 5 (GSE29013¹⁶), which showed more than a 32 fold-difference in expression levels. We observed similar batch effects in our testing cohort (FIG. 1e). After application of COMBAT, the batch effects were eliminated (FIG. 1b, d, f).

Analysis of NSCLC Survival Distributions Suggests Multiple Genes Govern Survival

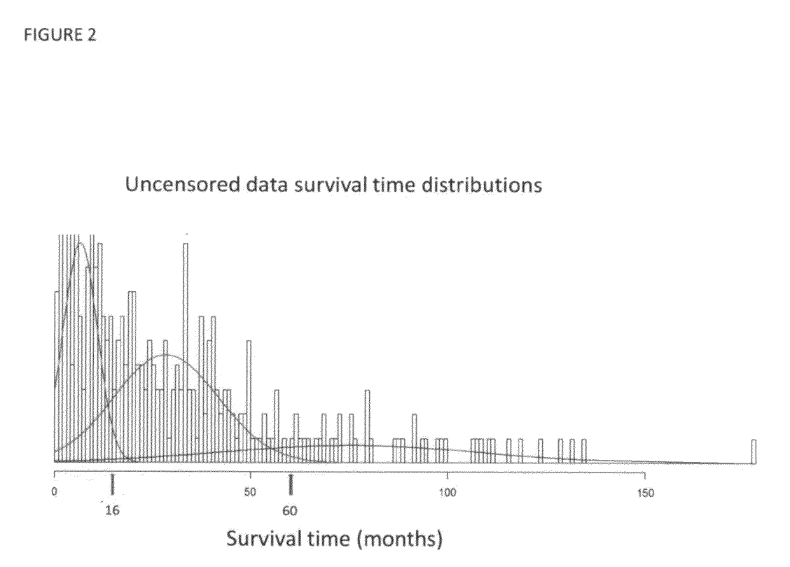

The overall survival (OS) of the 306 NSCLC patients that died before the studies concluded exhibited a three-peak distribution. We were able to fit data to three normal distributions and sort patients into three different groups: good outcome (>60 months), intermediate outcome (16-60 months), and poor outcome (<16 months; FIG. 2). The distributions suggested that OS was influenced by multiple genes, and consequently, we predicted there might be at least six or more genes that could be used to model OS.

Differential Gene Expression Analysis Yields Seven-Gene Score

To generate a multi-gene model for OS, we sought relevant genes using the

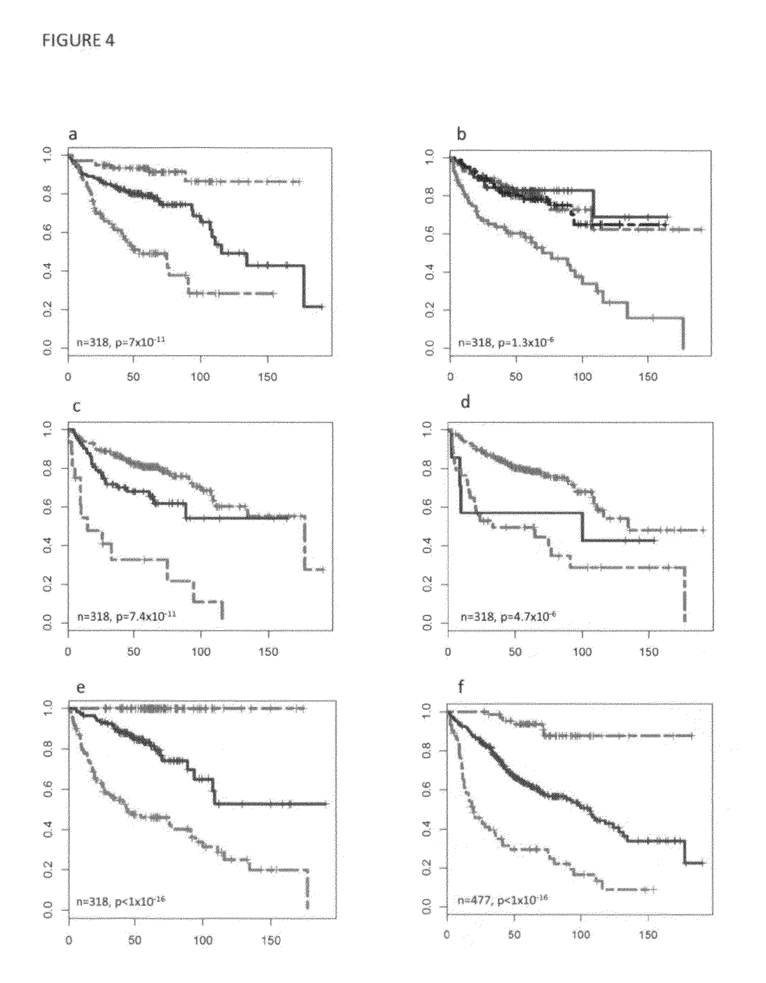

Siggenes in R, and compared the samples in our training cohort (n=1073; FIG. 3). Most of the studies from which we obtained our datasets used the tissues surrounding the lung tumors from the NSCLC patients (N) as a control as opposed to the more difficult to obtain normal lung tissues from the healthy lung (H). When we compared H and N, we found that there were 2555 of genes differentially expressed between H and N. This indicated that the tissue surrounding lung tumors was very different molecularly from actual healthy tissue. For comparison to cancerous lung tissue (Ca), the best control should be H and not N. However, we were restricted by the available data as many samples (170) in our datasets were surrounding tissue (N), and only 60 samples were healthy tissue samples (H). Thus, we employed an alternative approach and we used both H and N as separate controls. If a biomarker for NSCLC survival is reliable, it should be consistently different in the comparisons H vs Ca and N vs Ca. Since multiple two-group comparisons may introduce errors, we further compared the three groups simultaneously, and then found the genes expression differences that were common to all comparisons. This comparison revealed the genes that were differentially expressed for lung cancer tumors, but this did not necessarily mean they were all related to survival. We then analyzed the different survival groups using a similar comparison, and overlaid the probes of interest from the first comparison (214 probes) with those from the second comparison (338 probes), and found 129 common probes that were differentially expressed among all groups. We conducted univariate, multivariate, and Kaplan-Meier analysis and found 7 significant genes (FIG. 3, Table b. The p values in univariate, multivariate, and Kaplan-Meier analysis were less than 0.05.). We generated a seven-gene score for each patient by adding the values of each coefficient (from multivariate coxph model) multiplied by its respective gene expression value (seven-gene score=b1*gene1+b2*gene2+ . . . +b7*gene7). In our training cohort, survival data with all clinical parameters was only available for 477 patient samples. To avoid any confounding effect of ACT, we excluded any patient that received ACT or an unknown treatment (n=159). Applying this score in Kaplan-Meier analysis, we separated patients (n=318) into distinct three groups by best cutoffs (FIG. 4a).

Seven-Gene Score, Age and Stage are Independent Predictors

Multivariate analysis of available clinical parameters (age, gender, stage and cell type) suggested that cancer age, stage, and cell type might be independent predictors of survival (Table c). However, Kaplan-Meier analyses using these factors were only able to separate the patient samples into two distinct groups (FIG. 4b-d). When we introduced the seven-gene score into our multivariate analysis of clinical parameters, we found that while age and stage remained independent, cell type was no longer significant. Furthermore, the hazard ratio (HR) and p-value indicate that the seven-gene score is the most powerful independent predictor (Table c).

Seven-Gene Score, Age and Stage Constitute LCPI

Having determined the seven-gene score, age and stage as independent predictors of OS, we were able to generate survival functions:

S(t)=e^{-λt} (4)

LCPI=λ=b1*gene1+b2*gene2+ . . . +b7*gene7+b8*age+b9*stage (5)

Where S(t) is the survival probability before time t; λ is HR; LCPI is the lung cancer prediction index; b₁ to b₉ are coefficients calculated from the data in our training cohort with coxph model, they are 0.45(VANGL1), 0.36(GNAI3), 0.30(CTSB), −0.44(ANKRD11, −0.49(ITPKB), 0.03(KIAA0101), 0.05(PLOD2), 0.03(age) and 0.69(stage) separately, and remain constant in all LCPI calculations; gene₁ to gene₇ are the log₂ values of GEP; age is the real age (# in years); and stage values are 0 to 3 (stage IA=0, stage IB˜IIB=1, stage IIIA˜IV=3). To output the LCPI, we input the expression values of the seven genes (gene1, gene2, gene3, etc. log2 values), as well as the age (# in years), and stage of the cancer (0 to 3). Using above function (5), we were able to calculate the LCPI score for any patient and predict his/her OS (function (4)). Lower LCPI corresponded with higher survival probability while higher scores correspond to lower probability of survival, and higher likelihood of death and cancer recurrence. The cutoff value was the same as that in training cohort for the data from the same platform. For the data from different platform, we adjusted it to the best cutoff.

We separated our training cohort (n=318) into three clearly distinct groups using LCPI (FIG. 4e). At ten years after surgery, the survival probability of the low risk group was 100%, and remained the same even after 15 years. In the intermediate risk group, the survival probability at 15 years was 53±10% (p<0.001). The survival probability of the high-risk group was less than 20% at 15 years. From the analysis of the training cohort, we are able to obtain the best cutoff values for each risk group, and then apply them to the testing cohorts as pre-specified cutoffs. For datasets obtained using different platforms, the best cutoff calculation was performed to obtain cutoff values for each risk group.

ACT Negatively Impacts OS for Low and Intermediate Risk Groups

To discern whether ACT influences OS, we included data from patients that received ACT or an unknown treatment and applied the LCPI (n=477). The fact that we observed similar separation of risk groups with or without patients treated with ACT or unknown confirmed that the exclusion does not affect the LCPI model's ability to assign patients to risk groups (FIG. 4f). At 15 years after surgery, we observed lower survival probabilities for both the low and intermediate risk groups, which were 80±5% and 30±10% (p<0.05), respectively. Comparing to the cohort that did not receive treatment after surgery, the cohort that included patients who received ACT or an unknown treatment showed significant decreases in survival probabilities for the low and intermediate risk groups (80±5% vs. 100%, p<0.001; 30±10% vs. 53±10%, p<0.05). This suggests the possibility that ACT may have a negative impact on individuals with low or intermediate risk, as determined by the LCPI.

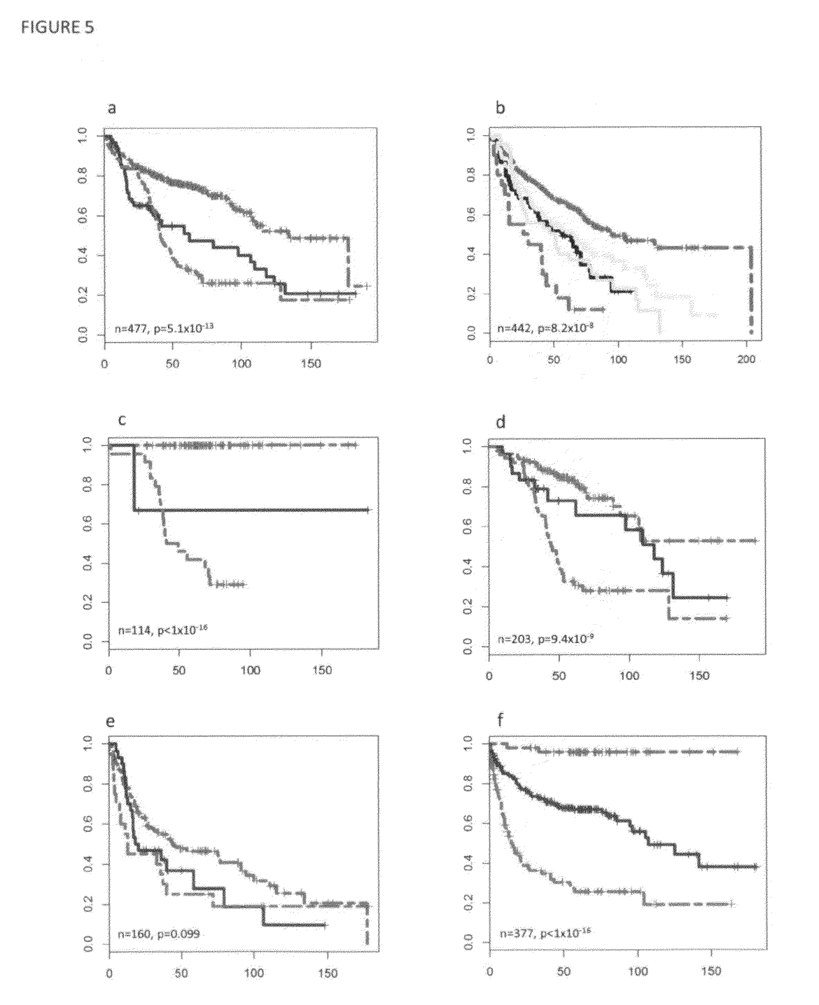

To further explore the impact of ACT on OS, we separated the patient pool (n=477) into non-ACT, ACT and unknown treatment groups. The non-ACT group exhibited the best OS, while the ACT group or surgery plus unknown treatment showed worse OS (FIG. 5a; p<0.001). We verified this outcome with the testing cohort (n=529) and observed similar results (FIG. 5b, p<0.001).

Given the effect we observed in the training and testing cohorts, we were curious whether ACT equally affected each LCPI risk group, so we analyzed the survival of each risk group in our training cohort separately. While ACT did not influence the survival of the patients in the high risk group, it was detrimental for patients in the low and intermediate risk groups (FIG. 5c-e).

Since OS may sometimes be influenced by other factors, we analyzed the RFS data as well. Recurrence after surgical resection is the main reason for the early death of NSCLC patients, and RFS is more reliable than OS. Recurrence data was only available for 377 of the 477 patients in our training cohort, and after application of LCPI, we were again able to distinguish the three risk groups (FIG. 5f; p<0.001). The recurrence data supports our analysis of the OS data.

Verification of LCPI in the Largest Multiple Institutions Dataset from USA and Canada

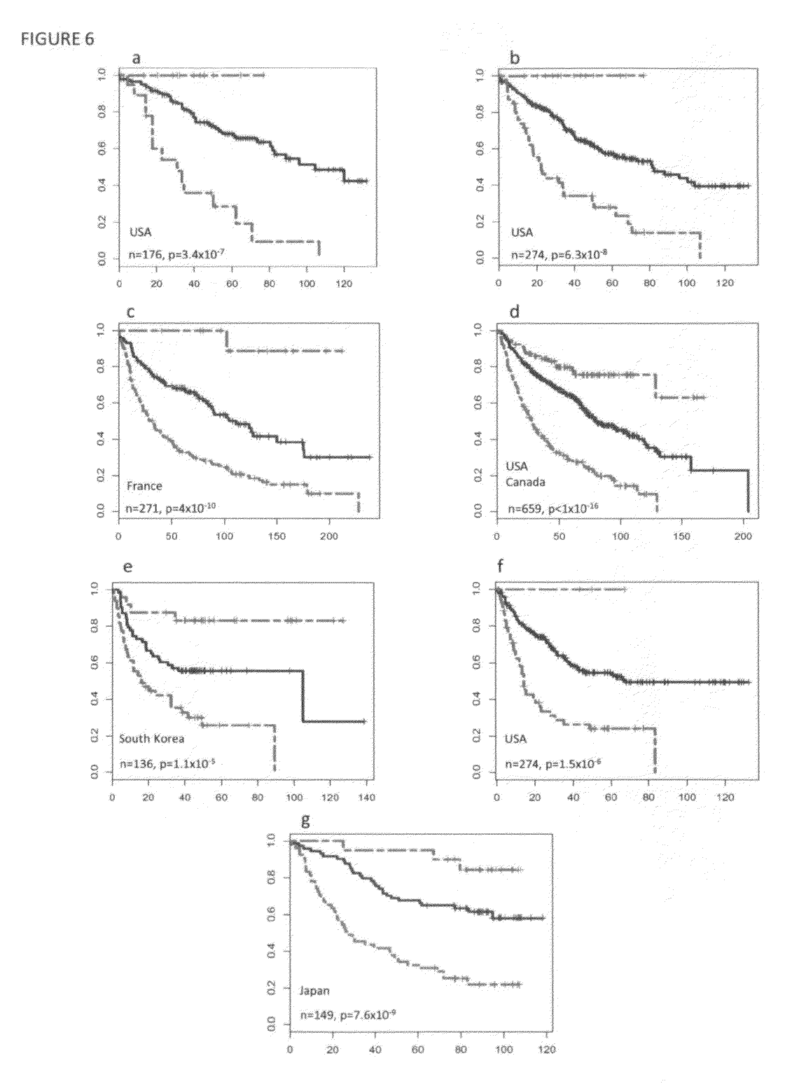

After integrating Jacob-00182⁹, GSE14814¹³ and GSE4573⁸ datasets with COMBAT, we produced the second largest multiple institutions dataset for NSCLC, which included all stages, three cell types and post-surgery ACT or ART from seven institutions in United States and Canada without batch effects (n=659). This dataset was obtained using the Affymetrix platform GPL96, which differed from our training cohort, so we verified the power of LCPI by adjusting it to the best cutoff. FIG. 6d showed that using besting cutoff values for this cohort performed using this platform, LCPI was able to separate the 659 NSCLC patients into three distinct risk subgroups. The OS probabilities in high risk subgroup at five years and 10 years were 28% and 9.5% respectively. All patients died before 130 months. The OS probabilities in intermediate risk subgroup at five years, 10 years and 15 years were 64%, 39% and 23%. The above results were very similar to the results in 477 of training dataset included ACT and unknown patients. But the OS probabilities in low risk subgroup at five years, 10 years and 15 years were 80%, 76% and 63% which were lower than that in 477 of training dataset. Given our previous analysis (FIG. 4-5), it is possible these that differences may be attributable to patients with ART and/or ACT (FIG. 5b). However, further study would be required to confirm the effect of post-surgical ACT for NSCLC. The above results indicated that LCPI was able to work in multiple institutions dataset of NSCLC including all stages, three cell types and different adjuvant treatments (ACT and/or ART).

Verification of LCPI in USA Dataset GSE42127

The samples in dataset GSE42127²¹ were from MD Anderson Cancer Center in Texas, United States. In this independent testing cohort, 133 patients were adenocarcinomas (ADC) and 43 patients were afflicted with squamous cell carcinomas (SCC). Forty-nine patients received ACT (mainly Carboplatin plus Taxanes) and 127 patients did not receive ACT. The patient sample included patients with cancer stages I, II, III and IV. We applied LCPI to this dataset, and since this cohort differed in platform, we used the best cutoff values to separate patients into different risk groups. FIG. 6a showed that LCPI was able to separate this cohort into three distinct subgroups (low, intermediate and high risk subgroups) similar to that in training cohort. The OS probability of low risk subgroup was up to 100% at 80 months, and the OS probability of intermediate risk subgroup was great than 40% at 10 years while all of the patients in high risk subgroup died before 10 years.

Verification of LCPI in the Largest Single Institution Dataset GSE41271 from USA

To date GSE41271²⁰, which included 176 samples from GSE42127²¹, was the largest NSCLC dataset from single institution in United States (n=275). The patients in this testing cohort belong to four different races (Caucasian, African American, Hispanic and Asian), and the clinical stages in this cohort were from IA to IV. There were 184 ADC patients, 80 SCC patients, and 10 patients that had five over rare cell types. One patient sample did not have the data necessary for analysis, and was not included. Using LCPI we performed Kaplan-Meier analyses for this testing cohort, which was performed with a different platform, by adjusting to the best cutoff. FIG. 6b showed that the results were very similar to that of the testing cohort GSE42127. The OS probability of low risk subgroup was up to 100% at 80 months, and the OS probability of intermediate risk subgroup was about 40% at 10 years while all of the patients in high risk subgroup died before 10 years. That suggested even in large dataset that included different races, some use of ACT, all stages and all cell types of NSCLC, LCPI still worked very well for identifying three different risk subgroups.

Verification of LCPI in the Largest Single Institution Dataset GSE30219 from France

GSE30219¹⁹ was the largest single institution dataset from France even excluding the control (n=14) and small cell lung cancer samples (n=22), which were not relevant to our study. There were 271 of NSCLC including all stages and seven cell types in this testing cohort. The data were obtained using the same platform as the training data, so we were able to apply LCPI to this cohort with pre-specified cutoff or the same cutoff value as that of the training cohort (6.83, 8.19). FIG. 6c showed that LCPI was able to separate this cohort into three distinct subgroups (low, intermediate and high risk subgroups) similar to that in training cohort and testing cohorts (GSE42127²¹, GSE41271²⁰). The OS probability of low risk subgroup was up to 100% at six years, stable at 89% from 10 years to over 18 years. The OS probability of intermediate risk subgroup was greater than 40% at 10 years and greater than 30% at 18 years. While the OS probabilities in high risk subgroup at any given time point were significantly lower than other two subgroups. This was a single dataset, and since we did not need to combine it with another, we did not perform COMBAT. Even without the use of COMBAT, LCPI still worked very well for identifying three different risk subgroups for the France dataset, which included all stages and all cell types of NSCLC.

Verification of LCPI to Predict RFS in South Korea Dataset GSE8894

Recurrences after surgical resection are the main reasons for the early deaths of NSCLC patients. RFS tends to be more reliable than OS because it is not affected by nonspecific deaths. If our LCPI model is reliable, it should work for both OS and RFS in multiple countries. This RFS dataset GSE8894¹⁰ from South Korea included 138 of NSCLC patients (two cell types). Two patients were missing the necessary data, and were thus excluded. The platform was the same as training cohort, but the stages information was not available. Then we applied LCPI without inputting data about cancer stage in 136 of NSCLC patients and defined risk groups by best cutoff. Although we did not have cancer stage information, our model was still able to define risk groups for the RFS data (FIG. 6e). The 136 of patients were separated into three different risk subgroups. All patients in high risk subgroup were recurrent before eight years while the probability of RFS in intermediate risk and low risk subgroups were great than 55% and 83% respectively at eight years.

Verification of LCPI to Predict RFS in the Largest Single Institution Dataset GSE41271 from USA

The largest NSCLC dataset for OS and RFS from a single institution in United States (n=275) was GSE41271²⁰. One patient sample did not possess the complete data required for analysis, and was excluded from our study. We applied LCPI to the 274 NSCLC patients in this cohort, which included RFS data from patients with all stage and all cell types. The cutoff value was the same as that for the OS analysis (FIG. 6b). LCPI separated the dataset into three significantly different risk subgroups (FIG. 6f). All patients in high risk subgroup experienced cancer recurrence before eight years while the probability of RFS in intermediate risk and low risk subgroups were great than 52% and 100% separately at five years. These results provide further support for the LCPI model's ability to separate low, intermediate and high risk subgroups for overall survival as well as recurrence datasets.

Verification of LCPI to Predict OS in Two-Channel Dataset GSE11969 from Japan

So far we have verified LCPI in all available NSCLC single channel array datasets from multiple countries. Some of datasets were performed with Agilent's two-channel array GPL7015 platform instead of single-channel array. There were 149 NSCLC patients in the Japanese cohort, GSE11969⁶, which included IA to IIIB and five cell types. Using function (3) we were able to transform two-channel array data into single channel data and get the LCPI score. Here we also defined risk group cutoffs to best cutoff. We showed that LCPI was able to separate this cohort into three different risk subgroups (FIG. 6g). The OS probabilities in the low, intermediate and high risk subgroups were 95%, 68% and 32% at five years and 84%, 58% and 22% at about 10 years respectively.

In summary, the most important aspect of any predictive model is its validation. To confirm the power of LCPI, we verified its ability to predict survival time using multiple datasets of NSCLC (n=1665, all stages and multiple cell types) from five countries (FIG. 6).

GSE42127 (n=176) and GSE41271 (n=274) included patients with all four stages and multiple cell types, some of which received ACT after operation. Application of LCPI to the OS data allowed us to separate these cohorts into the same risk groups we observed in the training cohort (FIG. 6a, b). We also analyzed the available RFS data (n=274) using LCPI. The recurrence analysis of the testing cohort further verified the predictive power of LCPI (FIG. 6f).

To assess whether LCPI can be accurately applied to data collected from different countries, we applied it to datasets GSE30219 (n=271, France), GSE8894 (n=136, South Korea), GSE11969 (n=149, Japan), and the combined datasets Jacob-00182, GSE14814 and GSE4573 (n=659, USA and Canada). After application of LCPI to the OS data of each dataset, we were able to observe distinct risk groups for all available testing cohorts (FIG. 6c, d, g). Similarly, we were able to predict the RFS for GSE8894 and separate patients into different risk groups (FIG. 6e). The fact that LCPI consistently predicted high, intermediate, and low risk groups for all the tested datasets demonstrates its reliability.

Discussion

We have proposed a multigene model (LCPI), which incorporates seven differentially expressed genes, age and stage, to predict clinical outcome. Utilizing the LCPI, we were able to separate patients into three distinct groups with different survival probabilities (FIG. 4, 6). Aided by this model, clinicians will be able to personalize post-surgical treatment for NSCLC patients. Low risk individuals have very high survival probabilities and may not require any further treatment beyond regular observation (FIG. 4e). The average age for patients that received surgery for NSCLC was around 62, and our model showed that the low risk individuals could survive more than 15 years after surgery. If we consider that the average world life expectancy is around 70-80 years old, then the average patient in the low risk group could expect to live out his/her full life expectancy after surgery. In fact, our data suggests that for patients in the low or intermediate risk groups, post-surgery treatment like ACT may actually decrease survival probabilities (FIG. 4e, f). For patients that have high risk, as determined by LCPI, surgery is insufficient. Based on the patient's survival probability, clinicians can determine whether to use conservative, aggressive, or experimental treatment strategies following surgical resection.

Efforts to find a predictive model for lung cancer have been underway since 2001⁴ and at present, more than 17 independent NSCLC gene expression datasets and their respective predictive models have been published. However, while these models span the spectrum between a single gene to hundreds of genes, their predictive abilities are limited by small samples sizes and institutional variations. In order to account for sample size and increase the power of our model, we combined nine different datasets with NSCLC samples and control samples for our training cohort. To account for institutional variation, we used COMBAT to completely eliminate the batch effects observed among the different datasets (FIG. 1). Using this strategy, we generated two of our largest datasets, a training cohort of n=1073 and a testing cohort of n=659. From the training cohort, we created a LCPI capable of predicting individual survival probabilities using the expression levels of seven genes, age, and stage. Since the success of a predictive model is determined by its verification, we tested our model using several independent datasets collected from multiple countries (FIG. 6). These testing cohorts contained samples from patients with multiple stages and cell types. The fact that our model was able to separate these patients into three distinct risk groups regardless of cancer stage, cell type, and country of origin, illustrates the exceptional reliability and predictive capacity of the LCPI.

Shedden et al. provided one of the largest gene-expression datasets for NSCLC in 2008⁹. After the analysis of several different methodologies for the prediction of tumor biology and the inference of patient survival, they concluded that the subject outcome was best predicted using 100 gene clusters with clinical parameters. In 2012, Okayama et al. proposed a similarly large predictive model using 174-gene signatures¹⁷. Regardless of predictive accuracy, however, the collection and analysis of hundreds of genes to infer patient prognosis is economically unfeasible and difficult to apply in practice. Furthermore, compared to many of published models for NSCLC, which have been developed from data truncated at 60 months, we've shown in our model verification that our seven-gene model is capable of clearly distinguishing patient survival groups from uncensored data collected over 200 months (FIG. 6c).

The postoperative use of ACT is the standard of care for the management of some stages of NSCLC. The benefits of ACT, however, remain debatable. Some studies have shown that NSCLC patients treated with ACT have prolonged survival²⁶⁻²⁸, while some of them failed to observe any overall survival benefit with ACT^{29,30}. Five of the largest adjuvant trials to date include: (1) National Cancer Institute of Canada (NCIC) JBR.10 (n=482), (2) Adjuvant Navelbine International Trialist Association (ANITA, n=840), (3) Big lung trial (BLT), (4) International Trialist Association Trial (IALT, n=1867), and (5) Adjuvant Lung Project Italy (ALPI)³¹. The NCIC JBR.10²⁶ and the ANITA trials²⁷ demonstrated OS benefit and the survival advantage did not diminish over time at seven years follow-up. The IALT showed a slightly improvement in the five-year survival rate of 4% with adjuvant chemotherapy³². The BLT^{29,33} and the ALPI³⁰ trials were negative. Another dataset of 2194 patients (1313 bevacizumab; 881 controls) from four phase II and III trials showed that bevacizumab significantly prolonged OS and RFS²⁸. The NSCLC Meta-analysis Collaborative Group published a paper in Lancet in April, 2010, which summarized 34 trials, showed the benefit of adjuvant therapy was undeniable at 5 years, the improvement was slight (4%) at 5 years³⁴. Contributing to the ongoing dialogue regarding the effectiveness of ACT, our analysis suggests that post-operative ACT treatment may have a detrimental effect on individuals that have low or intermediate risk, as determined by LCPI (FIG. 4e, f). While further investigation is necessary to confirm our observation, it highlights a pressing need to determine the effectiveness of ACT as a treatment for low-risk NSCLC. In some cases, postoperative treatment is unnecessary, and an accurate predictive model can help clinicians individualize treatments for NSCLC.

We conclude that survival time of NSCLC is a quantitative trait. The seven genes, age and stages together determine the survival probability at 10 and 15 years. LCPI is able to simultaneously define three risk subgroups for all stages and multiple cell types of NSCLC. Based on our analysis of patients defined to be low risk by LCPI, surgical resection may be sufficient to maximize overall survival and recurrence free survival, they were surgical curable.

### REFERENCES

1 Jemal, A. et al. Global cancer statistics. CA: a cancer journal for clinicians 61, 69-90, doi:10.3322/caac.20107 (2011).
2 Ramalingam, S. S. et al. Lung cancer: New biological insights and recent therapeutic advances. CA: a cancer journal for clinicians 61, 91-112, doi:10.3322/caac.20102 (2011).
3 Patel, M. I. & Wakelee, H. A. Adjuvant chemotherapy for early stage non-small cell lung cancer. Frontiers in oncology 1, 45, doi:10.3389/fonc.2011.00045 (2011).
4 Bhattacharjee, A. et al. Classification of human lung carcinomas by mRNA expression profiling reveals distinct adenocarcinoma subclasses. Proceedings of the National Academy of Sciences of the United States of America 98, 13790-13795, doi:10.1073/pnas.191502998 (2001).
5 Bild A H, et al. Oncogenic pathway signatures in human cancers as a guide to targeted therapies. Nature 439(7074):353-7 (2006).
6 Takeuchi, T. et al. Expression profile-defined classification of lung adenocarcinoma shows close relationship with underlying major genetic changes and clinicopathologic behaviors. Journal of clinical oncology: official journal of the American Society of Clinical Oncology 24, 1679-1688, doi:10.1200/JCO.2005.03.8224 (2006).
7 Gruber, M. P. et al. Human lung project: evaluating variance of gene expression in the human lung. American journal of respiratory cell and molecular biology 35, 65-71, doi:10.1165/rcmb.2004-02610C (2006).
8 Raponi, M. et al. Gene expression signatures for predicting prognosis of squamous cell and adenocarcinomas of the lung. Cancer research 66, 7466-7472, doi:10.1158/0008-5472.CAN-06-1191 (2006).
9 Director's Challenge Consortium for the Molecular Classification of Lung, A. et al. Gene expression-based survival prediction in lung adenocarcinoma: a multi-site, blinded validation study. Nature medicine 14, 822-827, doi:10.1038/nm.1790 (2008).
10 Lee, E. S. et al. Prediction of recurrence-free survival in postoperative non-small cell lung cancer patients by using an integrated model of clinical information and gene expression. Clinical cancer research: an official journal of the American Association for Cancer Research 14, 7397-7404, doi:10.1158/1078-0432.CCR-07-4937 (2008).
11 Kuner, R. et al. Global gene expression analysis reveals specific patterns of cell junctions in non-small cell lung cancer subtypes. Lung cancer 63, 32-38, doi:10.1016/j.lungcan.2008.03.033 (2009).
12 Lu, T. P. et al. Identification of a novel biomarker, SEMA5A, for non-small cell lung carcinoma in nonsmoking women. Cancer epidemiology, biomarkers & prevention: a publication of the American Association for Cancer Research, cosponsored by the American Society of Preventive Oncology 19, 2590-2597, doi:10.1158/1055-9965.EP1-10-0332 (2010).
13 Zhu, C. Q. et al. Prognostic and predictive gene signature for adjuvant chemotherapy in resected non-small-cell lung cancer. Journal of clinical oncology: official journal of the American Society of Clinical Oncology 28, 4417-4424, doi:10.1200/JCO.2009.26.4325 (2010).
14 Hou, J. et al. Gene expression-based classification of non-small cell lung carcinomas and survival prediction. PloS one 5, e10312, doi:10.1371/journal.pone.0010312 (2010).
15 Sanchez-Palencia, A. et al. Gene expression profiling reveals novel biomarkers in nonsmall cell lung cancer. International journal of cancer. Journal international du cancer 129, 355-364, doi:10.1002/ijc.25704 (2011).
16 Xie, Y. et al. Robust gene expression signature from formalin-fixed paraffin-embedded samples predicts prognosis of non-small-cell lung cancer patients. Clinical cancer research: an official journal of the American Association for Cancer Research 17, 5705-5714, doi:10.1158/1078-0432.CCR-11-0196 (2011).
17 Okayama, H. et al. Identification of genes upregulated in ALK-positive and EGFR/KRAS/ALK-negative lung adenocarcinomas. Cancer research 72, 100-111, doi:10.1158/0008-5472.CAN-11-1403 (2012).
18 Botling, J. et al. Biomarker discovery in non-small cell lung cancer: integrating gene expression profiling, meta-analysis, and tissue microarray validation. Clinical cancer research: an official journal of the American Association for Cancer Research 19, 194-204, doi:10.1158/1078-0432.CCR-12-1139 (2013).
19 Rousseaux, S. et al. Ectopic activation of germline and placental genes identifies aggressive metastasis-prone lung cancers. Science translational medicine 5, 186ra166, doi:10.1126/scitranslmed.3005723 (2013).
20 Sato, M. et al. Human lung epithelial cells progressed to malignancy through specific oncogenic manipulations. Molecular cancer research: MCR 11, 638-650, doi:10.1158/1541-7786.MCR-12-0634-T (2013).
21 Tang, H. et al. A 12-gene set predicts survival benefits from adjuvant chemotherapy in non-small cell lung cancer patients. Clinical cancer research: an official journal of the American Association for Cancer Research 19, 1577-1586, doi:10.1158/1078-0432.CCR-12-2321 (2013).
22 Wilkerson, M. D. et al. Differential pathogenesis of lung adenocarcinoma subtypes involving sequence mutations, copy number, chromosomal instability, and methylation. PloS one 7, e36530, doi:10.1371/journal.pone.0036530 (2012).
23 Chen, C. et al. Removing batch effects in analysis of expression microarray data: an evaluation of six batch adjustment methods. PloS one 6, e17238, doi:10.1371/journal.pone.0017238 (2011).
24 Chen, T., et al. Low-risk identification in multiple myeloma using a new 14-gene model. European journal of haematology 89, 28-36, doi:10.1111/j.1600-0609.2012.01792.x (2012).
25 Arriagada, R. et al. Long-term results of the international adjuvant lung cancer trial evaluating adjuvant Cisplatin-based chemotherapy in resected lung cancer. Journal of clinical oncology: official journal of the American Society of Clinical Oncology 28, 35-42, doi:10.1200/JCO.2009.23.2272 (2010).
26 Winton, T. et al. Vinorelbine plus cisplatin vs. observation in resected non-small-cell lung cancer. The New England journal of medicine 352, 2589-2597, doi:10.1056/NEJM0a043623 (2005).
27 Douillard, J. Y. et al. Adjuvant vinorelbine plus cisplatin versus observation in patients with completely resected stage IB-IIIA non-small-cell lung cancer (Adjuvant Navelbine International Trialist Association [ANITA]): a randomised controlled trial. The Lancet. Oncology 7, 719-727, doi:10.1016/S1470-2045(06)70804-X (2006).
28 Soria, J. C. et al. Systematic review and meta-analysis of randomised, phase II/III trials adding bevacizumab to platinum-based chemotherapy as first-line treatment in patients with advanced non-small-cell lung cancer. Annals of oncology: official journal of the European Society for Medical Oncology/ESMO 24, 20-30, doi:10.1093/annonc/mds590 (2013).
29 Waller, D. et al. Chemotherapy for patients with non-small cell lung cancer: the surgical setting of the Big Lung Trial. European journal of cardio-thoracic surgery: official journal of the European Association for Cardio-thoracic Surgery 26, 173-182, doi:10.1016/j.ejcts.2004.03.041 (2004).
30 Scagliotti, G. V. & Novello, S. Adjuvant therapy in completely resected non-small-cell lung cancer. Current oncology reports 5, 318-325 (2003).
31 Patel, M. I. & Wakelee, H. A. Adjuvant chemotherapy for early stage non-small cell lung cancer. Front Oncol 1, 45 (2011).
32 Arriagada, R. et al. Cisplatin-based adjuvant chemotherapy in patients with completely resected non-small-cell lung cancer. N Engl J Med 350, 351-60 (2004).
33 Brown, J. et al. Assessment of quality of life in the supportive care setting of the big lung trial in non-small-cell lung cancer. J Clin Oncol 23, 7417-27 (2005).
34 NSCLC Meta-analysis Collaborative Group. Adjuvant chemotherapy, with or without postoperative radiotherapy, in operable ono-small-cell lung cancer: two meta-analysis of individual patient data. The Lancet 375, 1267-1277 (2010).

TABLE 1
The Name, Gene ID, Location and Aliases of 91 Genes

| Name | Gene ID | Gene Location | Aliases |
|---|---|---|---|
| AGO4 | 192670 | 1 | EIF2C4 |
| ANKRD11 | 29123 | 16 | ANCO-1, ANCO1, LZ16, T13 |
| ANKRD40 | 91369 | 17 | |
| AP1S1 | 1174 | 7 | AP19, CLAPS1, EKV3, MEDNIK, SIGMA1A, AP1S1 |
| ATXN1L | 342371 | 16 | hCG_1646491, BOAT, BOAT1, ATXN1L |
| BEX5 | 340542 | X | GHc-351F8.1, NGFRAP1L1, BEX5 |
| BPHL | 670 | 6 | BPH-RP, MCNAA, VACVASE, BPHL |
| CBX8 | 57332 | 17 | PC3, RC1, CBX8 |
| CEP55 | 55165 | 10 | RP11-30E16.2, C10orf3, CT111, URCC6, CEP55 |
| CHPF | 79586 | 2 | UNQ651/PRO1281, CHSY2, CSS2, CHPF |
| CLIC5 | 53405 | 6 | RP11-546O15.1, MST130, MSTP130, CLIC5 |
| CST3 | 1471 | 20 | ARMD11, CST3 |
| CSTA | 1475 | 3 | AREI, STF1, STFA, CSTA |
| CTSB | 1508 | 8 | APPS, CPSB, CTSB |
| CTSD | 1509 | 11 | CLN10, CPSD, HEL-S-130P, CTSD |
| DHX37 | 57647 | 12 | DDX37, DHX37 |
| DNAJC27 | 51277 | 2 | RBJ, RabJS, DNAJC27 |
| DVL1 | 1855 | 1 | RP5-89003.5, DVL, DVL1L1, DVL1P1, DVL1 |
| EMP2 | 2013 | 16 | XMP, EMP2 |
| FAM111B | 374393 | 11 | hCG_1729960, CANP, POIKTMP, FAM111B |
| FCRLA | 84824 | 1 | FCRL, FCRL1, FCRLM1, FCRLX, FCRLb, FCRLc1/2, FCRLd, FCRLe, FCRX, FREB, FCRLA |
| FPR1 | 2357 | 19 | FMLP, FPR, FPR1 |
| GLT25D1 | 79709 | 19 | PSEC0241, GLT25D1, COLGALT1 |
| GLT25D2 | 23127 | 1 | RP11-498P10.2, C1orf17, GLT25D2, COLGALT2 |
| GNAQ | 2776 | 9 | RP11-494N1.1, CMC1, G-ALPHA-q, GAQ, SWS, GNAQ |
| GTDC1 | 79712 | 2 | Hmat-Xa, mat-Xa, GTDC1 |
| GVIN1 | 387751 | 11 | GVIN1, GVIN1P, VLIG-1, VLIG1, GVINP1 |
| HDAC4 | 9759 | 2 | AHO3, BDMR, HA6116, HD4, HDAC-4, HDAC-A, |
| HDAC5 | 10014 | 17 | HD5, NY-CO-9, HDAC5 |
| INCENP | 3619 | 11 | INCENP |
| INPP5A | 3632 | 10 | RP11-288G11.1, 5PTASE, INPP5A |
| IPMK | 253430 | 10 | IPMK |
| ITPK1 | 3705 | 14 | ITRPK1, ITPK1 |
| ITPR1 | 3708 | 3 | ACV, CLA4, INSP3R1, IP3R, IP3R1, SCA15, SCA16, SCA29, ITPR1 |
| JUNB | 3726 | 19 | AP-1, JUNB |
| KDSR | 2531 | 18 | DHSR, FVT1, SDR35C1, KDSR |
| KIAA0101 | 9768 | 15 | L5, NS5ATP9, OEATC, OEATC-1, OEATC1, PAF, PAF15, p1(PAF), p15/PAF, p15PAF |
| KLF8 | 11279 | X | RP13-1021K9.1, BKLF3, ZNF741, KLF8 |
| KLHL6 | 89857 | 3 | KLHL6 |
| LPAR1 | 1902 | 9 | RP11-104M22.2, EDG2, GPR26, Gpcr26, LPA1, Mrec1.3, VZG1, edg2, rec.1.3 |
| LRRFIP1 | 9208 | 2 | FLAP-1, FLAP1, FLIIAP1, GCF-2, GCF2, HUFI-1, TRIP, LRRFIP1 |
| MAD2L1 | 4085 | 4 | HSMAD2, MAD2, MAD2L1 |
| MARVELD | 91862 | 16 | MARVD3, MRVLDC3, MARVELD3 |
| MLL | 4297 | 11 | ALL-1, CXXC7, HRX, HTRX1, GAS7, MLL1, MLL1A, TET1-MLL, TRX1, WDSTS, KMT2A |
| MPZL1 | 9019 | 1 | RP1-313L4.1, MPZL1b, PZR, PZR1b, PZRa, PZRb, MPZL1 |
| MYLIP | 29116 | 6 | RP1-13D10.1, IDOL, MIR, MYLIP |
| MYOZ1 | 58529 | 10 | CS-2, FATZ, MYOZ, MYOZ1 |
| NCAPG | 64151 | 4 | CAPG, CHCG, NY-MEL-3, YCG1, NCAPG |
| NCOA2 | 10499 | 8 | GRIP1, KAT13C, NCoA-2, SRC2, TIF2, bHLHe75, NCOA2 |
| NCOA3 | 8202 | 20 | ACTR, AIB-1, AIB1, CAGH16, CTG26, KAT13B, RAC3, SRC-3, SRC3, TNRC14, TNRC16 |
| NPAL3 | 57185 | 1 | RP3-462O23.3, DJ462O23.2, NPAL3, NIPAL3 |
| OSTM1 | 28962 | 6 | HSPC019, GIPN, GL, OPTB5, OSTM1 |
| PKM2 | 5315 | 15 | CTHBP, HEL-S-30, OIP3, PK3, PKM2, TCB, THBP1, PKM |
| PLOD2 | 5352 | 3 | LH2, TLH, PLOD2 |
| PLOD3 | 8985 | 7 | LH3, PLOD3 |
| PPP1R15A | 23645 | 19 | GADD34, PPP1R15A |
| PPP4C | 5531 | 16 | PP4, PP4C, PPH3, PPP4, PPX, PPP4C |
| PRICKLE2 | 166336 | 3 | EPM5, PRICKLE2 |
| PTGR1 | 22949 | 9 | RP11-16L21.1, LTB4DH, PGR1, ZADH3, PTGR1 |
| PTPN5 | 84867 | 11 | PTPSTEP, STEP, PTPN5 |
| RALGPS2 | 55103 | 1 | RP4-595C2.1, dJ595C2.1, RALGPS2 |
| RBM12B | 389677 | 8 | MGC: 33837, RBM12B |
| RBM17 | 84991 | 10 | RP11-414H17.9, SPF45, RBM17 |
| RGS10 | 6001 | 10 | RGS10 |
| RGS19 | 10287 | 20 | GAIP, RGSGAIP, RGS19 |
| RGS2 | 5997 | 1 | GIG31, G0S8, RGS2 |
| RIC8A | 60626 | 11 | RIC8, RIC8A |
| RIPK1 | 8737 | 6 | RIP, RIP1, RIPK1 |
| RNF166 | 115992 | 16 | RNF166 |
| RP2 | 6102 | X | DELXp11.3, NM23-H10, NME10, TBCCD2, XRP2, RP2 |
| RUFY3 | 22902 | 4 | RIPX, SINGAR1, RUFY3 |
| SEL1L3 | NA | 4 | Sel-1L3, SEL1L3 |
| SH2D1B | 117157 | 1 | EAT2, SH2D1B |
| SLC7A8 | 23428 | 14 | LAT2, LPI-PC1, SLC7A8 |
| SPI1 | 6688 | 11 | hCG_25181, OF, PU.1, SFPI1, SPI-1, SPI-A, SPI1 |
| SPTAN1 | 6709 | 9 | EIEE5, NEAS, SPTA2, SPTAN1 |
| TADA3 | 10474 | 3 | ADA3, NGG1, STAF54, TADA3L, hADA3, TADA3 |
| TBX21 | 30009 | 17 | T-PET, T-bet, TBET, TBLYM, TBX21 |
| THAP8 | 199745 | 19 | THAP8 |
| THOC4 | 10189 | 17 | ALY, ALY/REF, BEF, REF, THOC4, ALYREF |
| TMED4 | 222068 | 7 | ERS25, HNLF, TMED4 |
| TRIM28 | 10155 | 19 | KAP1, RNF96, TF1B, TIF1B, TRIM28 |
| UBE2O | 63893 | 17 | E2-230K, UBE2O |
| VPS37D | 155382 | 7 | WBSCR24, VPS37D |
| ZNF331 | 55422 | 19 | RITA, ZNF361, ZNF463, ZNF331 |

TABLE 2
The Name, Gene ID, Location and Aliases of 10 Genes

| Name | GeneID | Location | Aliases |
|---|---|---|---|
| AMBP | 259 | Chromosome 9 | A1M, EDC1, HCP, HI30, |
| | | | IATIL, ITI, ITIL, ITILC, UTI |
| CALM1 | 801 | Chromosome 14 | CALML2, CAMI, CPVT4, |
| | | | DD132, PHKD, caM |
| CSTB | 1476 | Chromosome 21 | CST6, EPM1, EPM1A, |
| | | | PME, STFB, ULD |
| GNAI3 | 2773 | Chromosome 1 | RP5-1160K1.2, 87U6, ARCND1 |
| ING3 | 54556 | Chromosome 7 | HSPC301, Eaf4, ING2, |
| | | | MEAF4, p47ING3, ING3 |
| MMP14 | 4323 | Chromosome 14 | MMP-X1, MT-MMP, |
| | | | MTMMP1, MT1, MMP, |
| | | | MT1, MMP, |
| PBK | 55872 | Chromosome 8 | CT84, HEL164, Nori-3, |
| | | | SPK, TOPK, PBK |
| PCNA | 5111 | Chromosome 20 | |
| PTBP3 | 9991 | Chromosome 9 | RP11-165N19.1, ROD1 |
| VANGL1 | 81839 | Chromosome 1 | KITENIN, LPP2, STB2, |
| | | | STBM2 |

TABLE a
Summary of 17 GEP Datasets of NSCLC

| | | | | | | | Survival | Survival | |
|---|---|---|---|---|---|---|---|---|---|
| | | | Number of | | | | probability | probability | |
| | | | genes used | | | | of low risk | of low risk | Data |
| | | First | in author's | | Cell | Training/ | group at | group at | truncated |
| Ref no. | GSE ID | author | model | Stages | types | test | 5 years | 15 years | at 5 years |
| 5 | 3141 | Bild A H | NA | NA | ADC, | Training | 68%± | NA | NA |
| | | | | | SCC | | | | |
| 6 | 11969 | Takeuchi | | I-III | ADC | Test | 78%± | NA | No |
| | | T | | | | | | | |
| 7 | 1643 | Gruber | Healthy | NA | NA | Training | NA | NA | NA |
| | | M P | | | | | | | |
| 8 | 4573 | Raponi M | 100 | I | SCC, | Test | NA | NA | Yes |
| | | | | | ADC | | | | |
| 9 | NA | Shedden | 100 | I-III | ADC | Test | 62%± | NA | Yes |
| | | K | | | | | | | |
| 10 | 8894 | Lee E S | 6 | I-III | ADC, | Test | 60%± | NA | No |
| | | | | | SCC | | | | |
| 11 | 10245 | Kuner R | 17 | I-III | ADC; | Training | NA | NA | No |
| | | | | | SCC | | | | |
| 12 | 19804 | Lu T P | 1 | I-IV | ADC | Training | 22%±; | NA | Yes |
| | | | | | | | 45% ± _60%± | | |
| 13 | 14814 | Zhu C Q | 15 | I-II | ADC, | Test | 90%± | NA | No |
| | | | | | SCC | | | | (9 years) |
| 14 | 19188 | Hou J | 17 | I-IV | ALL | Training | 58% ± _68%± | NA | No |
| | | | | | | | | | (>10 years) |
| 15 | 18842 | Sanchez- | 92 | I-IV | ADC, | Training | NA | NA | No |
| | | Palencia | | | SCC | | | | |
| | | A | | | | | | | |
| 16 | 29013 | Xie Y | 59 | I | ADC, | Training | 46% ± _51%± | NA | Yes |
| | | | | | SCC | | | | (7 years) |
| 17 | 31210 | Okayama | 9 | I-II | ADC | Training | 84%± | NA | Yes |
| | | H | | | | | | | (98-2008) |
| 18 | 37745 | Botling J | 14(1) | NA | ADC, | Training | 61%± | 20%± | No |
| | | | | | SCC, LCC | | | | (95-2005) |
| 19 | 30219 | Rousseaux | 26 | I-IV | ALL | Test | 66%± | NA | Yes (Max: |
| | | S | | | | | | | 240 M) |
| 20 | 41271 | Sato M | 171 | I-III | ADC, | Test | 70%± | NA | No |
| | | | | | SCC | | | | |
| 21 | 42127 | Tang H | 18(12) | I-III | ADC | Test | 78%± | NA | No |
| | | | | | | | | | (96-2007) |

TABLE b
The Name, ID, Location and Aliases of Seven Common Genes

| Name | Gene ID | Location | Aliases |
|---|---|---|---|
| ANKRD11 | 29123 | Chromosome 16, | ANCO-1, ANCO1, LZ16, T13 |
| | | NC_000016.10 | |
| | | (89267619..89490561, | |
| | | complement) | |
| CTSB | 1508 | Chromosome 8, | APPS, CPSB |
| | | NC_000008.11 | |
| | | (11842524..11868137, | |
| | | complement) | |
| GNAI3 | 2773 | Chromosome 1, | RP5-1160K1.2, 87U6, ARCND1 |
| | | NC_000001.11 | |
| | | (109548564..109595843) | |
| ITPKB | 3707 | Chromosome 1, | IP3-3KB, IP3K, IP3K-B, IP3KB, |
| | | NC_000001.11 | PIG37 |
| | | (226631690..226739327, | |
| | | complement) | |
| KIAA0101 | 9768 | Chromosome 15, | L5, NS5ATP9, OEATC, OEATC- |
| | | NC_000015.10 | 1, OEATC1, PAF, PAF15, |
| | | (64364994..64387687, | p15(PAF), p15/PAF, p15PAF |
| | | complement) | |
| PLOD2 | 5352 | Chromosome 3, | LH2, TLH |
| | | NC_000003.12 | |
| | | (146069439..146161495, | |
| | | complement) | |
| VANGL1 | 81839 | Chromosome 1, | KITENIN, LPP2, STB2, STBM2 |
| | | NC_000001.11 | |
| | | (115641953..115698224) | |

TABLE c
Multivariate analysys of clinical data with/without
seven-gene score for OS (n = 318)

| | Without seven-gene | | With seven-gene | |
|---|---|---|---|---|
| | score | | score | |
| | | p (log-rank | | p (log-rank |
| Variables | HR | test) | HR | test) |
| Gender | 1.33 | 0.195 | | |
| Age | 1.04 | 0.0257 | 1.03 | 0.0496 |
| Stages (Coef) | 1.99 | 1.13 × 10⁻⁸ | 2.03 | 5.95 × 10⁻⁸ |
| Cell types (Coef) | 2.05 | 0.0261 | 1.58 | 0.1684 |
| Seven-gene score | | | 2.61 | 1.91 × 10⁻¹⁰ |
| (Coef) | | | | |

HR: hazard ratio;
Coef: coefficient

What is claimed is:

1. A gene expression panel, sequence or array indicative of overall and recurrence free survival time of a subject diagnosed with NSCLC (including any stages, any cell types), said panel or array consisting of primers or probes or sequences capable of measuring expression levels of a statistically significant number of one or more of the genes identified in Table 1 disclosed herein.

2. A gene expression panel, sequence or array indicative of overall survival time of a subject diagnosed with NSCLC (including any stages, any cell types), said panel or array consisting of primers or probes or sequences capable of measuring expression levels of a statistically significant number of one or more of the genes identified in Table 2 disclosed herein.

3. The gene expression panel, sequence or array according to claims 1 and 2, consisting of primers or probes or sequences capable of detecting one or more genes identified in one or more of the genes in Tables disclosed herein.

4. A diagnostic/prognostic kit containing sequences, probes or primers for measuring the expression of one or more genes identified in one or more of the Tables disclosed herein with or without one or more clinical parameters (age, stage, et al).

5. A method of diagnosing or prognosis or assessing a subject's susceptibility to develop NSCLC comprising:
a. extracting RNA from a biological sample of said subject containing cancer cells;
b. generating cDNA from said RNA;
c. amplifying said cDNA with probes or primers for genes or gene expression products, wherein said genes or gene expression products are selected from a statistically significant number of genes or gene expression products of one or more genes identified in one or more of the Tables disclosed herein;
d. obtaining from said amplified cDNA a profile of the expression levels of the selected genes or gene expression products in said sample; and
e. diagnosing or assessing a subject's prognosis upon a variance in the obtained profile of expression levels of the said selected genes or gene expression products in said subject's sample from the same selected genes or gene expression products of a control gene expression profile from a similar biological sample of a healthy subject, or diagnosing or assessing a subject's prognosis upon a similarity in the obtained profile of expression levels of said selected genes or gene expression products in said subject's sample to the same selected genes or gene expression products in a gene expression profile characteristic of a subject with NSCLC.

6. The method according to claim 5, wherein the variance in the obtained profile of expression levels of the said selected genes or gene expression products (including RNA and/or protein) in said subject's sample is used to determine whether a subject is at a low, intermediate, or high risk of NSCLC with or without one or more clinical parameters (age, stage, et al).

7. The method of claim 5, wherein the variance in the obtained profile of expression levels of the said selected genes or gene expression products (including RNA and/or protein) in said subject's sample can be used to determine the type of treatment that the subject should receive with or without one or more clinical parameters (age, stage, et al).

8. The method of claim 5, for treating NSCLC in an individual by modulating expression of one or more genes identified in one or more of the Tables disclosed herein; thereby altering differential expression of the NSCLC genes to treat the individual.

9. The method of claim 5, wherein the variance in the obtained profile of expression levels of the said selected genes or gene expression products (including RNA and/or protein) can be either upregulated or downregulated as compared to a control.

10. A method of diagnosing or assessing a subgroup of NSCLC in a subject, the method comprising:
i. extracting RNA from a biological sample of said subject containing cancer cells;
ii. generating cDNA from said RNA;
iii. amplifying said cDNA with probes or primers for genes or gene expression products, wherein said genes or gene expression products are selected from one or more genes identified in one or more of the Tables disclosed herein;
iv. obtaining from said amplified cDNA a profile of the expression levels of the selected genes or gene expression products in said sample; and
v. diagnosing or assessing a subject's subgroup based upon a variance in the obtained profile of expression levels of the said selected genes or gene expression products in said subject's sample from the same selected genes or gene expression products of a control gene expression profile from a similar biological sample of a healthy subject, or diagnosing or assessing a subject's subgroup based upon a similarity in the obtained profile of expression levels of said selected genes or gene expression products in said subject's sample to the same selected genes or gene expression products in a gene expression profile characteristic of a subject with NSCLC.

11. The method of claim 10, wherein the profile of the expression levels of the genes is used to compute a statistically significant value based on differential expression of the group of genes, wherein the computed value correlates to a diagnosis for a subgroup of NSCLC.

12. The method of claim 10, wherein the subgroups of NSCLC are low, intermediate and high risk subgroups with or without one or more clinical parameters (age, stage, et al).

13. A method of assessing a subject's susceptibility to develop NSCLC, the method comprising: amplifying cDNA or detect protein from a biological sample containing lung tissue and/or blood samples of the subject to obtain expression levels of a statistically significant number of genes or gene expression products (including RNA and/or protein) obtained from said sample, wherein said genes or gene expression products are selected from a statistically significant number of genes or gene products of Table 1 or Table 2, thereby assessing a subject's susceptibility to develop NSCLC based on a change in a profile of expression levels between said selected genes or gene products (including RNA and/or protein) of said sample from the same selected genes or gene products of a control healthy expression profile, wherein said change indicates a subject's susceptibility to develop NSCLC.

14. The method according to claim 13, wherein said change is an increase in expression level of one or more genes or gene products (including RNA and/or protein) of said profile.

15. The method according to claim 13, wherein said change is a decrease in expression level of one or more genes or gene products (including RNA and/or protein) of said profile.

16. The method according to claim 13, wherein said control expression profile is a gene expression profile or RNA sequence from a similar biological sample of a healthy subject.

17. The method according to claim 13, wherein said control expression profile is a gene expression profile or RNA sequence from a biological sample of a subject with NSCLC.
